(19) United States
(12) Patent Application Publication
(10) Pub. No.: US 2024/0366747 A1
(43) Pub. Date: Nov. 7, 2024

(54) UNIVERSAL VACCINE FOR INFLUENZA VIRUS BASED ON TETRAMERIC M2 PROTEIN INCORPORATED INTO NANODISCS

(71) Applicant: The Board of Trustees of the University of Illinois, Urbana, IL (US)

(72) Inventors: Federico A. Zuckermann, Champaign, IL (US); Stephen G. Sligar, Urbana, IL (US)

(73) Assignee: The Board of Trustees of the University of Illinois, Urbana, IL (US)

(21) Appl. No.: 18/574,482

(22) PCT Filed: Jul. 14, 2022
(86) PCT No.: PCT/US2022/073717

§ 371 Date: Dec. 27, 2023

Related U.S. Application Data
(60) Provisional application No. 63/222,672, filed on Jul. 16, 2021.

(51) Int. Cl. A61K 39/145; A61K 39/00; A61K 39/39; A61P 31/16; C12N 7/00

(52) U.S. Cl. CPC A61K 39/145; A61K 39/39; A61P 31/16; C12N 7/00; A61K 2039/5252; A61K 2039/552; A61K 2039/55516; A61K 2039/55555; A61K 2039/55561; A61K 2039/572; A61K 2039/575; C12N 2760/16122; C12N 2760/16134

(57) ABSTRACT

Immunogenic compositions that include a full-length influenza A virus matrix 2 (M2) protein, an amphipathic molecule, and at least one phospholipid, which assemble to form a nanodisc, are described. Use of the immunogenic compositions, for example as a universal influenza virus vaccine, is described.

### CROSS REFERENCE TO RELATED APPLICATIONS

This application claims the benefit of U.S. Provisional Application No. 63/222,672, filed Jul. 16, 2021, which is incorporated herein by reference in its entirety.

### ACKNOWLEDGMENT OF GOVERNMENT SUPPORT

This invention was made with government support under Grant Nos. NI17HFPXXXXXG032 and 2016-31100-06017 awarded by the United States Department of Agriculture and Grant No. R35 GM118145 awarded by the National Institutes of Health. The government has certain rights in the invention.

### FIELD

This disclosure concerns immunogenic compositions that include a nanodisc containing full-length, tetrameric influenza A virus matrix 2 (M2) protein, and use of the compositions as universal influenza virus vaccines.

### BACKGROUND

The syndrome resulting from the infection of swine with influenza A virus (IAV) presents a major economic burden to the pork industry. Vaccination is the most efficient and cost-effective means to prevent and control influenza in swine populations. Whole inactivated virus (WIV) vaccines are the most common type of biologic used in the U.S. to protect pigs against IAV. This type of vaccine relies primarily on the effective stimulation of virus neutralizing antibodies against the surface glycoprotein hemagglutinin (HA). The HA is a type I glycoprotein that forms homotrimers where each monomer consists of two disulfide-linked HA1 and HA2 subunits after cleavage of the HA0 precursor. Antibodies that inhibit virus hemagglutination of red blood cells are considered correlates of protection. Since WIV vaccines primarily elicit a humoral immune response, virus variants that are unrecognized by the vaccine-induced anti-HA antibodies render the vaccination ineffective. Experimental data indicates that the protection provided to swine by commercial WIV influenza vaccines against contemporary swine IAV (swIAV) is limited, which is due in part to the heterologous antigenic nature of the constantly increasing diversity of swine IAV. Thus, the substantial diversity among co-circulating IAV in swine herds poses a significant challenge for effective vaccine development. Since 2009, the genetic diversity of swIAV in pig populations has expanded globally, generating additional pandemic threats such as the novel variant H3N2v swIAV that infected more than 330 humans in the United States from 2011-2013. The intense IAV surveillance in pigs has revealed that the transmission of IAV from humans to swine is far more frequent than swine-to-human zoonosis, and it is central in seeding swine globally with new viral diversity (Nelson and Vincent, Trends Microbiol 23(3):142-153, 2015). Hence, there is an urgent need to develop effective IAV vaccines and vaccination strategies conferring broad-spectrum protection to reduce the economic losses to the pork industry as well as to mitigate the risk of the emergence of re-assorted IAV in swine with the potential to become zoonotic.

### SUMMARY

Provided herein are immunogenic compositions that include a full-length influenza A virus matrix 2 (M2) protein, an amphipathic molecule, and at least one phospholipid. As described herein, the M2 protein, the amphipathic molecule and the at least one phospholipid self-assemble to form a nanodisc (ND). In some implementations, the full-length M2 protein is a pandemic (PDM) or triple reassortant internal gene (TRIG) M2 protein or a variant thereof. The amphipathic molecule can be, for example, a protein or polypeptide (such as a membrane scaffold protein-MSP), an organic polymer, or a natural or synthetic nucleic acid molecule. In some implementations, the at least one phospholipid includes a glycerophospholipid, an ether glycerophospholipid, or a sphingophospholipid. In some examples, the immunogenic composition further includes an adjuvant, which is optionally incorporated into the nanodisc.

Also provided herein are methods of eliciting an immune response against influenza A virus in a subject. In some implementations, the method includes administering to the subject an effective amount of an immunogenic composition disclosed herein. In some implementations, the immunogenic composition is administered in combination with another influenza virus vaccine, such as a vaccine that includes whole inactivated influenza virus. In some examples, the immune response includes both cell-mediated and humoral immune responses. In some examples, the subject is porcine or human.

The foregoing and other objects and features of the disclosure will become more apparent from the following detailed description, which proceeds with reference to the accompanying figures.

### BRIEF DESCRIPTION OF THE DRAWINGS

FIG. 1: SDS-PAGE/Western blot analysis of the M2 protein incorporated into nanodiscs (ND). The sample was purified on a Ni-NTA column followed by size-exclusion chromatography. Lane 1: the M2:ND sample was mixed with Laemmli sample buffer containing 50 mM DTT and incubated at ambient temperature for 10 minutes, then run on a 4-20% Tris/HCl gradient gel (BioRad), transferred to a 0.2 μm PVDF membrane and probed with anti-His-tag antibody (mouse monoclonal 33D10.D2.G8 IgG1 kappa, Rockland). Lane 2: Pre-stained protein standards were used for molecular weight reference, from top: 105, 82, 47, 33, 27 and 17.5 kDa.

FIG. 2: Levels of serum and respiratory mucosal antibody induced upon vaccination of swine using either the M2:ND vaccine or an influenza WIV vaccine. A group of 4-week-old pigs (n=4) were immunized with M2:ND intramuscularly (IM) twice at a two-week interval. A second group was immunized IM with FLUSURE XP®, a commercial tetravalent swine influenza WIV vaccine. A third group was mock-vaccinated. Serum was obtained from animals immediately before the first IM immunization (pre) and ten days after the second immunization (post) and tested by ELISA for the presence of IgG specific for IAV, M2 or M2 ectodomain (M2e). To detect IAV- or M2e-specific antibodies, the ELISA plates were coated with inactivated whole influenza virus (Influenza virus) or a synthetic M2e peptide (M2e), respectively. To detect M2-specific antibodies (M2), recombinant M2 protein was bound to the wells of an ELISA plate precoated with anti-His-tag antibody, which captured M2 via the His-tag present in the recombinant M2 protein. After reacting the wells with the respective serum, the presence of IgG was detected using HRP-labeled rabbit anti-porcine IgG antibodies.

FIG. 3: Frequency of interferon-γ-secreting T cells induced upon vaccination of swine using either the M2:ND vaccine or an influenza WIV vaccine. Peripheral blood mononuclear cells were isolated from the same groups of pigs described in FIG. 2 ten days after the second immunization. The cells were stimulated for 8 hours with either an inactivated whole influenza virus expressing the pdmM2 gene (Influenza virus) or purified recombinant M2 protein. Cells producing interferon-γ in response to the stimuli were enumerated using an interferon-γ-ELISPOT.

FIG. 4: Viral load in lung lavage fluid (LLF) of pigs five days after challenge (CHL) with swIAV H3N2 isolate NY11 (red H3 antigenic cluster) or isolate MN16 (green H3 antigenic cluster). To attain maximum whole inactivated virus (WIV) vaccine-induced protection, groups of four-week-old pigs were immunized with a NY11-based WIV vaccine (N=5) and challenged with NY11. A control group (N=4) received a mock vaccine. The vaccine was administered intramuscularly (IM) twice, at a two-week interval, and the pigs were challenged two weeks later with NY11. To test the ability of the M2:NDs to increase the breath of protective immunity, groups of pigs were immunized with the NY11 based WIV vaccine either alone (N=8) or in combination with M2:ND (N=8), and challenged with MN16. A third group was vaccinated only with M2:NDs (N=8), and a fourth group was mock vaccinated (N=4). All four groups were challenged two weeks after the second vaccination with MN16. A strict control group (N=4) was neither vaccinated or challenged. LLF was collected five days after virus challenge and the load of infectious virus determined using a plaque formation assay. Each symbol represents the number of plaque-forming units per ml (PFU/ml) of LLF from a single pig. Also shown is the mean±SE of PFU/ml for each group. Statistical differences between treatment groups were determined by unpaired t-test. **P<0.01; ****P<0.0001.

FIG. 5: Viral load in LLF of pigs five days after challenge with swIAV isolate MN16. Groups of pigs (N=7) were vaccinated twice 26-days apart with NDs displaying the swine influenza virus M2 protein (M2:ND) or bare-NDs without M2 protein (mock vaccine). A third group (N=2) was only challenged. Animals were euthanized 5 days after challenge with the H3N2 swIAV strain MN16. LLF was collected and analyzed for the presence of infectious virus using a plaque formation assay. Each symbol represents the number of plaque forming units per ml (PFU/ml) of LLF from a single pig. Also shown is the mean±SE of PFU/ml of each group. Statistical differences between treatment groups were determined by unpaired t-test. *P<0.05.

### SEQUENCE LISTING

The Sequence Listing is submitted as an ST.26 Sequence Listing XML file, named SeqList.xml, created on Jul. 5, 2022, having a size of 59,957 bytes, which is incorporated by reference herein. In the accompanying sequence listing:
SEQ ID NOs: 1-8 are amino acid sequences of exemplary IAV M2 proteins.
SEQ ID NO: 9 is the amino acid sequence of a protein tag.
SEQ ID NO: 10 is the amino acid sequence of an M2 protein N-terminal domain.
SEQ ID NO: 11 is the amino acid sequence of an M2 protein transmembrane helix.
SEQ ID NO: 12 is the amino acid sequence of an M2 protein amphipathic helix.
SEQ ID NO: 13 is the amino acid sequence of an M2 protein C-terminal domain.
SEQ ID NO: 14 is the amino acid sequence of a full-length M2 protein with a His tag.
SEQ ID NOs: 15-26 are amino acid sequences of helical domains.
SEQ ID NOs: 27-52 are amino acid sequences of exemplary membrane scaffold proteins.
SEQ ID NOs: 53 and 54 are amino acid sequences of influenza virus antigenic motifs.
SEQ ID NOs: 55-58 are amino acid sequences of exemplary protein tags.

### DETAILED DESCRIPTION

### I. Abbreviations

ADCC antibody-dependent cellular cytotoxicity
ApoA1 apolipoprotein A1
BALF bronchoalveolar lavage fluid
CMI cell-mediated immunity
ELISA enzyme-linked immunosorbent assay
HA hemagglutinin
HRP horse-radish peroxidase
IAV influenza A virus
IFN interferon
IM intramuscular
IN intranasal
IS immune stimulatory
LLF lung lavage fluid
M2 matrix protein 2
M2e matrix protein 2 ectodomain
MLPA monophosphorylated lipid A
NA neuraminidase
ND nanodisc
PDM pandemic
swIAV swine influenza A virus
TCID tissue culture infectious dose
TEV tobacco etch virus
TLR toll-like receptor
TRIG triple-reassortant internal gene
WIV whole inactivated virus

### II. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In order to facilitate review of the disclosure, the following explanations of specific terms are provided:

Adjuvant: A substance or vehicle used to enhance antigenicity, for example antigenicity of the disclosed nanodiscs. Adjuvants can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Other types of emulsion adjuvants can include water-oil-water emulsions. Polyphosphazene polymers can also be used to create nanoparticles and used either alone (Schulze et al., Nanomedicine 13(7):2139-2178, 2017), or in combination with other immunostimulatory molecules, such as polyI:C and a host defense peptide (HDP) as in the polyphosphazene triple adjuvant combination (TriAdj; see, e.g., U.S. Pat. No. 9,061,001; and Garg et al., Vaccine 35 (35 Pt A):4486-4489, 2017), which induces robust immune responses when delivered either intramuscularly or intranasally (Chand et al., Front Bioeng Biotechnol 9:625482, 2021). In addition, immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, polyI:C, or PolyICLC. Additional adjuvants for use in the disclosed immunogenic compositions include the QS21 purified plant extract, Matrix M, AS01, MF59, and ALFQ adjuvants. A variety of adjuvants are known in the art (see, e.g., Singh (ed.) Vaccine Adjuvants and Delivery Systems. Wiley-Interscience, 2007). Adjuvants can be used in combination with the disclosed immunogenic compositions, for example to enhance their immunogenicity.

Administration: The introduction of a composition, such as an immunogenic composition, into a subject by a chosen route. Administration can be local or systemic. Exemplary routes of administration include, but are not limited to, oral, injection (such as subcutaneous, intramuscular, intradermal, intraperitoneal, intravenous, and intratumoral), sublingual, rectal, transdermal (for example, topical), intranasal, vaginal, and inhalation routes.

Amphipathic molecule: A molecule having both hydrophilic and hydrophobic properties. In some implementations herein, the amphipathic molecule is a protein, such as a membrane scaffold protein (e.g., an apolipoprotein or derivative thereof).

Apolipoprotein A1 (Apo-A1): A protein that is the primary component of high-density lipoprotein (HDL) particles. The Apo-A1 protein is encoded by the APOA1 gene. Nucleotide and protein sequences of Apo-A1 from a variety of species are publicly available. For example, human sequences can be found under NCBI Gene ID 335 (e.g., GENBANK™ Accession Nos. NM_000039.3 and NP_000030.1 are exemplary mRNA and protein sequences, respectively) and porcine sequences can be found under NCBI Gene ID 397691 (e.g., GENBANK™ Accession Nos. NM_214398.1 and NP_999563.1 are exemplary mRNA and protein sequences, respectively).

Conservative variant: A protein containing conservative amino acid substitutions that do not substantially affect or decrease the function of a protein, such as an influenza virus M2 protein or a membrane scaffold protein. “Conservative” amino acid substitutions are those substitutions that do not substantially affect or decrease a function of a protein, such as the ability of the protein to elicit an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some implementations less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of a protein, such as an M2 protein, such as the ability to elicit an immune response when administered to a subject. For instance, if an amino acid residue is essential for a function of the protein, even an otherwise conservative substitution may disrupt that activity. Thus, a conservative substitution does not alter the basic function of a protein of interest.

Effective amount: A quantity of a specific substance, such as an immunogenic composition disclosed herein, sufficient to achieve a desired effect, such as an immune response in a subject. For instance, the effective amount can be the amount necessary to decrease the amount of IAV present in an infected subject, or it may be the amount necessary to prevent or inhibit infection in a subject that is not currently infected. A “prophylactically effective amount” refers to an amount of an agent or composition that inhibits or prevents establishment of an infection, such infection by IAV. It is understood that to obtain a protective immune response against a pathogen of interest, multiple administrations of a disclosed immunogenic composition, and/or administration of a disclosed immunogenic composition as the “prime” in a prime boost protocol, wherein the boost immunogen can be different from the prime immunogen, may be required. Accordingly, an effective amount of a disclosed immunogenic composition can be the amount of the composition sufficient to elicit a priming immune response in a subject that can be subsequently boosted with the same or a different immunogen (such as whole inactivated influenza virus) to generate a protective immune response. In some implementations, a therapeutically effective amount is the amount necessary to decrease IAV viral load in an infected subject by at least 10%, at least 20%, at least 50%, at least 75%, at least 80%, at least 90%, at least 95%, or even 100%, for example as compared to viral load prior to treatment. In other implementations, an effective amount is the amount necessary to decrease the risk of contracting an IAV infection in a healthy subject by at least 10%, at least 20%, at least 50%, at least 75%, at least 80%, at least 90%, at least 95%, or even 100%, such as compared to a non-vaccinated subject. In yet other implementations, the effective amount can be an amount that decreases virus shedding in nasal secretions, decreases lung pathology, decreases loads of virus in serum and lung and/or decreases shedding of virus upon challenge with HA mismatched virus (such as H3N2), such as by at least 10%, at least 20%, at least 50%, at least 75%, at least 80%, at least 90%, at least 95%, or even 100%, such as compared to a non-vaccinated subject.

Ether glycerophospholipid: A peroxisome-derived glycerophospholipid characterized by an alkyl chain attached to the sn-1 position by an ether bond (as opposed to an ester bond in standard glycerophospholipids) (see, e.g., Dean and Lodhi, Protein Cell 9(2):196-206, 2018).

Glycerophospholipid: A glycerol-based phospholipid found, for example, in biological membranes. The term “glycerophospholipid” refers to any derivative of glycerophosphoric acid that contains at least one O-acyl, O-alkyl, or O-(1-alkenyl) group attached to the glycerol residue (IUPAC, Compendium of Chemical Terminology, 2^{nd} ed., Compiled by A. D. McNaught and A. Wilkinson, Blackwell Scientific Publications, Oxford, 1997). Glycerophospholipids include, for example, phosphatidie acid, phosphatidylethanolamine, phosphatidylcholine, phosphatidylserine, phosphatidylinositol. phosphatidylinositol phosphate. phosphatidylinositol bisphosphate, and phosphatidylinositol trisphosphate.

Glycolipid: A lipid with a carbohydrate attached by a glycosidic (covalent) bond. The role of glycolipids is to maintain the stability of the cell membrane and to facilitate cellular recognition, which is important for the immune response and in the connections that allow cells to connect to one another to form tissues. alpha-Galactosylceramide (α-GalCer, KRN7000) is a synthetic glycolipid derived from structure-activity relationship studies of galactosylceramides isolated from the marine sponge Agelas mauritianus. α-GalCer is a potent activator of invariant natural killer T (iNKT) cells, and a model CD1d antigen. The invariant T cell receptor of the iNKT cell is able to bind the CD1d:glycolipid complex leading to iNKT cell activation in both mice and humans. In combination with a peptide antigen. α-GalCer is able to stimulate a strong immune response against the epitope. The CD1d:glycolipid:TCR interaction activates the iNKT cell which can then activate the dendritic cell. This causes the release of a range of cytokines and licenses the dendritic cell to activate a peptide-specific T cell response. This adjuvant acts through this cellular interaction, rather than through classic pattern recognition receptor pathways.

Heterologous: Originating from a separate genetic source or species. For example, a heterologous polypeptide or polynucleotide refers to a polypeptide or polynucleotide derived from a different source or species.

Immune response: A response of a cell of the immune system, such as a B cell, T cell, or monocyte, to a stimulus. In some implementations, the response is specific for a particular antigen (an “antigen-specific response”), such as an influenza virus M2 protein. In some implementations, the immune response is a T cell response, such as a CD4+ response or a CD8+ response. In other implementations, the response is a B cell response, and results in the production of specific antibodies. “Priming an immune response” refers to treatment of a subject with a “prime” immunogen/immunogenic composition to induce an immune response that is subsequently “boosted” with a boost immunogen/immunogenic composition. Together, the prime and boost immunizations produce the desired immune response in the subject. A “humoral immune response” refers to an immune response mediated by antibodies produced by B cells. A “cell-mediated immune response” refers to an immune response mediated by the activation of phagocytes and cytotoxic T cells, and the release of cytokines in response to an antigen.

Immunogenic composition: A composition comprising an immunogen that elicits an immune response, such as a measurable T cell or B cell response (such as production of antibodies) against an antigen (for example, IAV M2 protein) included on the immunogen or encoded by a nucleic acid molecule included in the immunogen. In one example, an immunogenic composition is a composition that includes a disclosed M2 protein, or nanodisc comprising an M2 protein, that induces a measurable CTL response against the M2 protein, or induces a measurable B cell response (such as production of antibodies) against the M2 protein, when administered to a subject. For in vivo use, the immunogenic composition typically will include the nanodisc in a pharmaceutically acceptable carrier and may also include other agents, such as an adjuvant (for example, TriAdj, a TLR agonist, MPLA or a CpG oligonucleotide). The immunogenic composition can also include one or more glycolipids, such as α-GalCer, which in some examples is incorporated directly into the M2:ND protein assembly.

Immunize: To render a subject protected from infection by a particular infectious agent, such as IAV. Immunization does not require 100% protection. In some examples, immunization provides at least 40%, at least 50%, at least 60%, at least 70%, at least 80%, at least 90% or at least 95% protection against infection compared to infection in the absence of immunization.

Influenza virus: A segmented negative-strand RNA virus that belongs to the Orthomyxoviridae family. There are three types of influenza viruses, A, B and C.

Influenza A virus (IAV): A negative-sense, single-stranded, segmented RNA virus, which has eight RNA segments (PB2, PB1, PA, NP, M, NS, HA and NA) that code for 11 proteins,

including RNA-directed RNA polymerase proteins (PB2, PB1 and PA), nucleoprotein (NP), neuraminidase (NA), hemagglutinin (subunits HA1 and HA2), the matrix proteins (M1 and M2) and the non-structural proteins (NS1 and NS2). This virus is prone to rapid evolution resulting from an error-prone polymerase and segment reassortment. The host range of influenza A is diverse, and includes humans, birds (e.g., chickens and aquatic birds), horses, marine mammals, pigs, bats, mice, ferrets, cats, tigers, leopards, and dogs. In animals, most influenza A viruses cause mild localized infections of the respiratory and intestinal tract. However, highly pathogenic influenza A strains, such as H5N1, cause systemic infections in poultry in which mortality may reach 100%. Animals infected with IAV often act as a reservoir for the influenza viruses and certain subtypes have been shown to cross the species barrier to humans.

Influenza A viruses can be classified into subtypes based on allelic variations in antigenic regions of two genes that encode surface glycoproteins, namely, hemagglutinin (HA) and neuraminidase (NA), which are required for viral attachment and cellular release. There are 18 different influenza A virus HA antigenic subtypes (H1 to H18) and 11 different influenza A virus NA antigenic subtypes (N1 to N11). H1-H16 and N1-N9 are found in wild bird hosts and may be a pandemic threat to humans. H17-H18 and N10-N11 have been described in bat hosts and are not currently thought to be a pandemic threat to humans.

Specific examples of IA V include, but are not limited to: H1N1 (such as 1918 H1N1), H1N2, H1N7, H2N2 (such as 1957 H2N2), H2N1, H3N1, H3N2, H3N8, H4N8, H5N1, H5N2, H5N8, H5N9, H6N1, H6N2, H6N5, H7N1, H7N2, H7N3, H7N4, H7N7, H7N9, H8N4, H9N2, H10N1, H10N7, H10N8, H11N1, H11N6, H12N5, H13N6, and H14N5.

In 2009, H1N1 influenza was the most common cause of human influenza. A new strain of swine-origin H1N1 emerged in 2009 and was declared pandemic by the World Health Organization. This strain was referred to as “swine flu.” H1N1 influenza A viruses were also responsible for the Spanish flu pandemic in 1918, the Fort Dix outbreak in 1976, and the Russian flu epidemic in 1977-1978.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include proteins, peptides, nucleic acids, or viruses that are at least 50% pure, such as at least 75%, 80%, 90%, 95%, 98%, 99%, or even 99.9% pure.

Matrix 2 (M2) protein: One of two matrix proteins found in influenza A viruses. M2 is a transmembrane protein that forms a small proton channel in the virus envelope. The M2 protein promotes uncoating of the IAV ribonucleoprotein (RNP) core after membrane fusion and promotes release of the viral RNP into the host cell cytoplasm. M2 also plays a role in viral assembly and release. Sequences of IAV M2 proteins are publicly available, such as in the Influenza Research Database (Zhang et al., Nucleic Acids Res 45(D1):D466-D74, 2017), and exemplary M2 protein sequences are set forth herein as SEQ ID NOs: 1-8. In the context of the present disclosure, a “full-length M2 protein” is an M2 protein that consists essentially of the complete M2 sequence of 97 amino acids, or lacks no more than 5, no more than 4, no more than 3, no more than 2 or no more than 1 amino acid relative to a wild-type M2 protein of 97 amino acids (e.g., SEQ ID NO: 1) or includes no more than 5, no more than 4, no more than 3, no more than 2, or no more than 1 additional amino acids relative to a wild-type M2 protein of 97 amino acids (e.g., SEQ ID NO: 1).

Membrane scaffold protein (MSP): An amphipathic helical protein that self-assembles with phospholipids to form nanoscale membrane bilayers having a discoidal, substantially discoidal or approximately discoidal shape, which is referred to as a nanodisc (see, e.g., Skar-Gislinge et al., J Am Chem Soc 132:13713-13722, 2010). MSPs can be naturally occurring membrane scaffold proteins, such as apolipoprotein A-1, or can be synthetic amphipathic proteins, such as synthetic MSPs based on ApoA1 or other apolipoproteins (e.g., ApoE3, ApoE4 or ApoCIII).

Nanodisc: A nanoscale particle composed of phospholipids and an encircling amphipathic helical belt protein, referred to as a membrane scaffold protein (MSP).

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds, molecules or agents (e.g., a nanodisc).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular implementations, to be suitable for administration to a subject, the carrier may be sterile, and/or suspended or otherwise contained in a unit dosage form containing one or more measured doses of the immunogenic composition suitable to elicit the desired anti-IAV immune response. The unit dosage form may be, for example, in a sealed vial that contains sterile contents or a syringe for injection into a subject, or lyophilized for subsequent solubilization and administration or in a solid or controlled release dosage.

Phospholipid: A class of amphiphilic lipids that have a hydrophilic “head” containing a phosphate group and two hydrophobic “tails” derived from fatty acids, which are joined by a glycerol molecule. Phospholipids include glycerophospholipids, which are the primary component of biological membranes, and sphingolipids (such as sphingomyelin).

Sphingophospholipid: A type of phospholipid containing a backbone of sphingoid bases. Examples of sphingophopholipids include, but are not limited to, ceramide and sphingomyelin.

Subject: Living multi-cellular vertebrate organisms, a category that includes both human and non-human animals, such as non-human mammals (such as birds, pigs, mice, rats, rabbits, sheep, horses, cows, bats and non-human primates, or any other animal that can be infected by an influenza virus).

Synthetic: Produced by artificial means in a laboratory, for example a synthetic nucleic acid or protein can be chemically synthesized in a laboratory.

Unit dosage form: A physically discrete unit, such as a capsule, tablet, or solution, that is suitable as a unitary dosage for a human or animal subject, each unit containing a predetermined quantity of one or more active ingredient(s) calculated to produce a therapeutic effect, in association with at least one pharmaceutically acceptable diluent or carrier, or combination thereof.

Vaccine: A preparation of immunogenic material capable of stimulating an immune response, administered for the prevention, amelioration, or treatment of infectious or other types of disease. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. Exemplary vectors include plasmids and viral vectors (such as adeno-associated viral vectors and lentiviral vectors).

### III. Introduction

Influenza A viruses (IAV) are one of the major infectious disease threats to the animal and human population due to both the health impact of annual influenza and the tremendous potential global consequences of influenza pandemics. In addition to the significant economic impact of influenza to pork producers, swIAV also represents a risk for the introduction of viruses into the human population resulting from antigenic shift. The most reliable approach to limiting IAV transmission within swine populations is through an appropriate vaccination program that protects against currently circulating genetic and antigenic diversity. Current IAV vaccines are based on strain-specific surface glycoprotein hemagglutinin (HA) antigens which are effective only when the predicted vaccine strains and circulating viruses are well-matched (Sandbulte et al., Vaccines 3(1):22-73, 2015). However, these vaccines have several limitations, including the emergence of antigenically novel viruses as a result of antigenic drift or shift. Antigenic drift refers to the frequent emergence of strains with different antigenicity, due to the ability of the virus to escape pre-existing immunity via point mutations in genes encoding HA and neuraminidase (NA). Antigenic shift involves the introduction of novel IAV strains with HA genes (from a zoonotic reservoir) that the human population has not previously experienced. Notably, the reverse direction also occurs, namely the introduction into swine of a triple-reassortant internal gene (TRIG) constellation in the late 1990s with a human origin H3 HA. The lack of pre-existing immunity in the animal or human population can enable the generation of novel viruses exhibiting antigenic shift with the potential to spread to a large percentage of the respective population, thus increasing pandemic risk. The current strategy of influenza vaccination does not prevent pandemic outbreaks, and protection efficacy is reduced or ineffective when re-assorted strains of IAV emerge resulting in antigenic shift in the HA antigens yielding a virus with the potential to become pandemic.

The present disclosure addresses disease prevention by vaccinating swine against IAV. The economic losses resulting from swine influenza rank among the top three health challenges to the pork industry. Protection of swine by commercial IAV whole inactivated virus (WIV) vaccines is limited in part due to co-circulation of multiple strains of antigenically distinct viruses in the same region. This disclosure aims to improve the breadth of coverage of WIV vaccines by developing an innovative biologic that relies upon a highly conserved viral protein (M2) displayed in its natural transmembrane configuration and in a stable form in nanodisc (ND) assemblies (M2:ND). Data disclosed herein demonstrates that the immunization of swine with M2:ND elicits the production of M2-specific antibodies that recognize virions. Further, the M2:ND vaccine also elicits IAV-specific interferon (IFN)-γ-secreting T cells, which is consistent with the presence of T-cell epitopes in the C-terminal domain of M2 (Deng et al., Vaccines 3(1):105-136, 2015). Data is also disclosed herein that shows immunization of swine with M2:ND alone provides protective immunity from challenge with influenza virus. Because a high degree of conservation exhibited by the M2 isoform present in the great majority of swIAV currently circulating in American swine herds, it is expected that the M2:ND vaccine will provide broad protective immunity as a stand-alone biologic.

### IV. Universal Influenza Virus Immunogenic Compositions and Use Thereof

The failure of currently available vaccines to protect against drifted seasonal influenza viruses or from antigenically novel pandemic viruses has driven the field towards the concept of a universal influenza vaccine that can overcome the phenomena of antigenic changes (Vincent et al., Vet Microbiol 206:35-44, 2017; Rajao and Vincent, ILAR J 56(1):44-52, 2015). Matrix protein 2 (M2) is a viroporin displayed on the influenza virus envelope as a tetramer that acts as an ion channel important for the uncoating of the virus upon cellular entry. M2 is a 97 amino acid single-pass transmembrane protein, which is known to oligomerize, forming a tetrameric proton-selective channel that comprises an integral component of the viral envelope. M2 can be divided into three parts: N-terminal extra-virion domain (residues 2-22), transmembrane domain (residues 23-46), and C-terminal intra-virion domain (residues 47-97). The intra-virion domain contains an amphipathic helix and a tail, which are involved in regulating virus assembly, virus budding, and the proton channel activity.

Prior to the present disclosure, studies focused on using only the extra-virion domain of M2 (M2e) as an immunogen for vaccine development. Unlike a number of M2e-based vaccines that are under various stages of development, the present disclosure describes the use of full-length M2 displayed in its natural transmembrane configuration as the vaccine antigen. This novel antigen design is made possible by incorporating M2 into special lipid nanoparticles, termed nanodiscs (ND). Nanodiscs are composed of soluble assemblies that incorporate transmembrane proteins into a native-like lipid bilayer, enabling the reconstitution of the native structure and function of transmembrane proteins into a highly stable and well characterized nanostructure. Because ND epitaxially display both sides of the membrane, both the extra-virion domain as well as the organizing intra-virion domains of M2 are effectively and stably displayed. By being inserted into the lipid bilayer of the ND, these nanostructures enable the reconstitution of M2 in its native full-length transmembrane configuration.

M2 is present in very few copies on the virus envelope (16-20 molecules/virion), and only the 22 amino acid-long ectodomain (M2e) of this molecule protrudes from the viral envelope. Natural M2e is poorly immunogenic, and as a result of virus infection, an antibody response against M2e is of low magnitude. Because the M2 protein is highly expressed on virus-infected cells but scantly present in virions, virus infected cells, rather than virions, are thought to be the target of M2e-based protective immunity. The mechanism of protection is believed to be largely accomplished by Fcγ-receptor mediated effector mechanisms involving, among other cells, alveolar macrophages, which by eliminating virus-infected cells, limit virus replication. While M2e is exclusively a target for humoral immunity, the C-terminal intra-virion domain of M2 has highly conserved T-cell epitopes (Deng et al., Vaccines 3(1):105-136, 2015), which are shared by various subtypes of IAV. Given that cell-mediated immunity (CMI) against influenza virus is known to contribute to accelerated virus clearance and restrict disease progression, CMI is an important component of a broadly protective immune response. Vaccination with full-length M2 is expected to elicit both humoral and cell-mediated immunity, leading to the development of a broadly protective immune response.

Provided herein are immunogenic compositions that include a full-length influenza A virus matrix 2 (M2) protein, an amphipathic molecule, and at least one phospholipid. The M2 protein, the amphipathic molecule and the at least one phospholipid self-assemble to form a nanodisc. The full-length M2 sequence can be, for example, a consensus sequence of the pandemic (PDM) or triple reassortant internal gene (TRIG) isoforms of swine influenza virus, or a variant thereof. In some implementations, the amino acid sequence of the full-length M2 protein is at least 80%, at least 85%, at least 90%, at least 95%, at least 96%, at least 97%, at least 98%, or at least 99% identical to any one of SEQ ID NOs: 1-8. In some examples, the amino acid sequence of the full-length M2 protein comprises or consists of any one of SEQ ID NOs: 1-8. In particular non-limiting examples, the amino acid sequence of the full-length M2 protein comprises or consists of SEQ ID NO: 1.

In some implementations, the full-length M2 protein is fused to a protein tag, such as a His tag or variant thereof. In some examples, His tag includes the amino acid sequence GHHHHHHIEGR (SEQ ID NO: 55) or GHHHHHHHDYDIPTTENLYFQG (SEQ ID NO: 56), and can optionally include an N-terminal methionine residue (for example, the tag of SEQ ID NO: 57 or SEQ ID NO: 58). In other examples, the protein tag is a Myc tag, a FLAG tag, or a hemagglutinin (HA) tag. In particular examples, the protein tag includes a protease cleavage site, such as a factor X (FX) cleavage site or a tobacco etch virus (TEV) protease cleavage site.

In some implementations, the amphipathic molecule includes a protein or polypeptide, such as a membrane scaffold protein (MSP). In some examples, the MSP is a derivative of human or porcine apolipoprotein A1 (Apo-A1), such as a truncated form of human or porcine Apo-A1. In particular examples, the amino acid sequence of the MSP is at least 80%, at least 85%, at least 90%, at least 95%, at least 96%, at least 97%, at least 98%, or at least 99% identical to any one of SEQ ID NOs: 27-52. In specific non-limiting examples, the amino acid sequence of the MSP comprises or consists of any one of SEQ ID NOs: 27-52. In one example, the amino acid sequence of the MSP comprises or consists of SEQ ID NO: 27. Additional MSPs are described in section V.

In other implementations, the amphipathic molecule comprises an organic polymer, or a natural or synthetic nucleic acid.

In some implementations, the at least one phospholipid includes a glycerophospholipid, an ether glycerophospholipid, or a sphingophospholipid (for example, sphingomyelin), or any combination of two or more thereof.

In some examples, the glycerophospholipid includes phosphatidyl choline, phosphatidyl ethanolamine, phosphatidyl serine, phosphatidyl inositol, cardiolipin, lysophospholipid, dipalmitoyl-phosphatidylcholine, dimyristoyl phosphatidyl choline, 1-palmitoyl-2-oleoyl-phosphatidyl choline, 1-palmitoyl-2-oleoyl-phosphatidyl serine, 1-palmitoyl-2-oleoyl-phosphatidyl ethanolamine, dihexanoyl phosphatidyl choline, dipalmitoyl phosphatidyl ethanolamine, dipalmitoyl phosphatidyl inositol, dimyristoyl phosphatidyl ethanolamine, dimyristoyl phosphatidyl inositol, dihexanoyl phosphatidyl ethanolamine, dihexanoyl phosphatidyl inositol, 1-palmitoyl-2-oleoyl-phosphatidyl ethanolamine, 1-palmitoyl-2-oleoyl-phosphatidyl inositol, or any combination thereof.

In other examples, the ether glycerophospholipid includes 1,2-di-O-phytanyl-sn-glycero-3-phosphocholine, 1,2-di-O-phytanyl-sn-glycero-3-phosphoethanolamine, 1,2-di-O-phytanyl-sn-glycerol, glycerol dialkyl glycerol tetraether, 1,2-di-O-octadecyl-sn-glycero-3-phosphocholine, 1,2-di-O-(9Z-octadecenyl)-sn-glycero-3-phosphocholine, 2-3-diphytanyl-O-sn-glycerol, caldarcheol, isocalarcheol, gentiobiosyl archaeol, archaetidylethanoloamine, gentyobiosyl caldarc haetidylethanoloamine, or any combination thereof.

In some implementations, the phospholipid further includes cholesterol, or another molecule or moiety that promotes stability of the phospholipid.

In some implementations, the immunogenic composition further includes an adjuvant, such as adjuvant suitable for parenteral or mucosal immunization (see, e.g., Freytag et al., Vaccine 23(15):1804-1813, 2005). In some examples, the adjuvant is incorporated into the nanodisc M2 assembly. In some examples, the adjuvant includes a toll-like receptor (TLR) agonist, such as a TLR4 agonist or a TLR9 agonist. In specific examples, the TLR4 agonists includes monophosphoryl lipid A (MPLA). In other specific examples, the TLR9 agonist includes a CpG oligonucleotide, such as a CpG oligonucleotide modified with cholesterol (for example, the cholesterol-anchored CpG D19). In yet other examples, the adjuvant includes a polyphosphazene, and optionally additional components such as polyI:C and a host defense peptide. In one example, the adjuvant includes polyphosphazene, polyI:C and a host defense peptide (such as in TriAdj). In another example, α-GalCer is incorporated into the lipid mixture as part of the nanodisc assembly. In another example, the adjuvant includes a polysaccharide molecule, such as delta inulin alone or modified with a CpG oligonucleotide, such as Advax-CpG55.2™, which is comprised of delta inulin polysaccharide particles formulated with CpG55.2, a toll-like receptor 9 (TLR9)-active oligonucleotide.

In some implementations, the immunogenic composition further includes a pharmaceutically acceptable carrier.

In some implementations, the immunogenic composition further includes whole inactivated influenza virus, or another suitable influenza virus vaccine, such as a seasonal influenza virus vaccine.

Also provided herein are methods of eliciting an immune response against influenza A virus in a subject. In some implementations, the method includes administering to the subject an effective amount of an immunogenic composition disclosed herein. In some examples, the subject has previously received an influenza virus vaccine, such as a seasonal influenza virus vaccine. In other examples, the subject is further administered an influenza virus vaccine, such as a seasonal influenza virus vaccine. In specific examples, the influenza virus vaccine (such as the seasonal influenza virus vaccine) includes whole inactivated virus. In some examples, the immune response includes both cell-mediated and humoral immune responses.

In some implementations of the disclosed methods, the subject is porcine. In other implementations, the subject is human.

### IV. Influenza A Virus M2 Proteins

Consensus amino acid sequences of the pandemic (PDM) and triple reassortant internal gene (TRIG) isoforms of swine influenza virus M2 (Gao et al., J Gen Virol 98:2001-2010, 2017) are provided below, along with variants of each sequence. Amino acid changes in each variant (relative to the respective consensus sequence) are indicated in bold underline.

Swine IAV M2 PDM Consensus Sequence
(SEQ ID NO: 1)
MSLLTEVETPTRSEWECRCSDSSDPLAIAANIIGILHLILWITDRLFFK
CIYRRFKYGLKRGPSTEGVPESMREEYQQEQQSAVDVDDGHFVNIELE
PDM P1 Variant
(SEQ ID NO: 2)
MSLLTEVETPTRSEWECRCSDSSDPLVIAANIIGILHLILWIADRLFFK
CIYRRFKYGLKRGPSTEGVPESMREEYQQEQQSAVDVDDGHFVNIELE
PDM P2 Variant
(SEQ ID NO: 3)
MSLLTEVETPTRSEWECRCSDSSDPLVIAANIIGILHLILWITDRLFFK
CIYRRFKYGLKRGPSTEGVPESMREEYQQEQQSAVDVDDGHFVNIELE
Swine IAV M2 TRIG Consensus Sequence
(SEQ ID NO: 4)
MSLLTEVETPIRNGWECKCNDSSDPLTDAASIIGILHLILWILDRLFFK
CIYRRFKYGLQRGPSTEGVPESMREEYRQKQQSAVDVDDGHFVNIVLE
TRIG T1 Variant
(SEQ ID NO: 5)
MSLLTEVETHINGWECKCNDSSDPLIDAASIIGILHLILWILDRLFFKC
IYRRFKYGLQRGPSTEGVPESMREEYRQKQQSAVDVDDGHFVNIVLE
TRIG T2 Variant
(SEQ ID NO: 6)
MSLLTEVETPIRSGWECKCNDSSDPLTDAASIIGILHLILWILDRLFFK
CIYRRFKHGLQRGPSTEGVPESMREEYRQEQQSAVDVDDGHFVNIVLE
TRIG T3 Variant
(SEQ ID NO: 7)
MSLLTEVETPTRNGWECKCNDSSDPLTDAASIIGILHLILWILDRLFFK
CIYRRFKYGLQRGPSTEGVPESMREEYRQKQQSAVDVDDGHFVNIVLE
TRIG T4 Variant
(SEQ ID NO: 8)
MSLLTEVETPIRNGWECRCNDSSDSLIDAASIIGILHLILWILDRLFFK
CIYRRFKYGLQRGPSTEGVPESMREEYRQKQQSAVDVDDGHFVNIVLE

In some implementations of the disclosed immunogenic compositions and methods, the full-length M2 protein is a consensus sequence of the PDM or TRIG isoforms of swine influenza virus, or a variant thereof. In some examples, the amino acid sequence of the full-length M2 protein is at least 80%, at least 85%, at least 90%, at least 95%, at least 96%, at least 97%, at least 98%, or at least 99% identical, or is 100% identical, to any one of SEQ ID NOs: 1-8. In particular non-limiting examples, the amino acid sequence of the full-length M2 protein comprises or consists of SEQ ID NO: 1.

### V. Membrane Scaffold Proteins (MSP)

In some implementations, nanodiscs are composed of phospholipids and two copies of an encircling amphipathic helical membrane scaffold protein (MSP) (see, e.g., Denisov and Sligar, Chem Rev 117:4669-4713, 2017; Denisov et al., Chapter 25 in Lipid-Protein Interactions: Methods and Protocols, Jörg H. Kleinschmidt (ed.), Methods in Molecular Biology, 2003; and U.S. Pat. No. 7,592,008, which are herein incorporated by reference). In some implementations of the present disclosure, the MSP is a naturally occurring MSP, such as an apolipoprotein, for example apolipoprotein A1, AII, CI, CII, CIII or E, or apolipophorin III. In other implementations, the MSP is an artificial MSP, such as an MSP derived from ApoA1, Apo AII, ApoCI, ApoCII, ApoCIII, ApoE, apolipophorin III, myoglobin or hemoglobin. Artificial MSPs can be modified by inserting additional helical domains (see Table 1, below), provided that modified MSPs retain the capacity to self-assemble (with phospholipid) into nanodiscs.

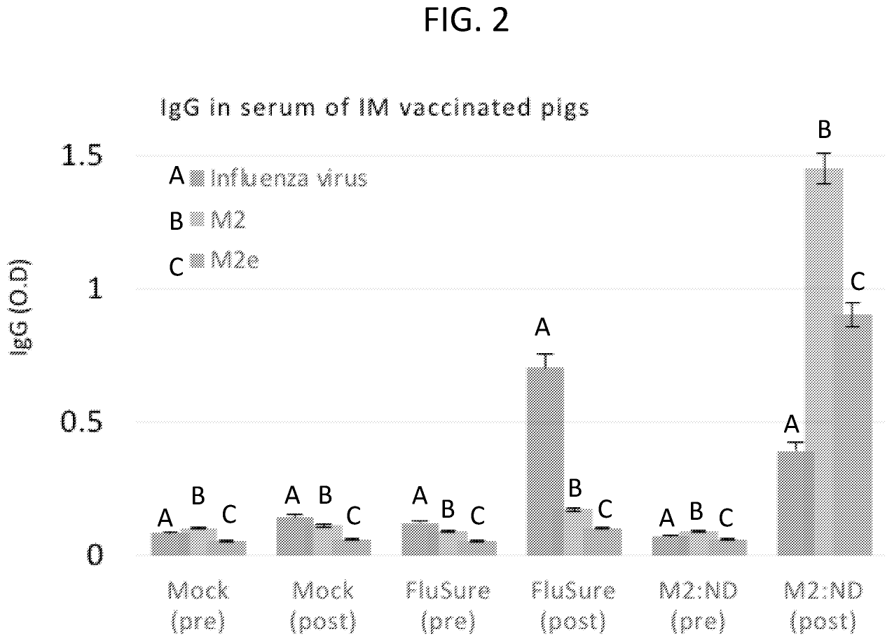

The MSPs of the present disclosure are amphipathic, with one portion of its structure hydrophilic and facing the aqueous solvent, and a second portion of its structure hydrophobic and facing the center of the hydrophobic bilayer that is to be stabilized. The elements of secondary structure of the protein generate the hydrophilic and hydrophobic regions in three dimensional space. In view of the necessity to be amphipathic, helical proteins are well-suited as MSPs. In some implementations, the MSPs disclosed herein have a helix as the fundamental amphipathic building block. Each MSP has an amino acid sequence which forms amphipathic helices with more hydrophobic residues (such as A, C, F, I, L, M, V, W or Y) predominantly on one face of the helix and more polar or charged residues (such as D, E, N, Q, S, T, H, K or R and sometimes C) on the other face of the helix (see FIG. 2 of U.S. Pat. No. 7,592,008, which is herein incorporated by reference). In some examples, the helical building block is periodically punctuated with residues (such as proline or glycine) that introduce flexibility into the overall structure by interrupting the general topology of the helix. In specific examples, these punctuations occur about every 20-25 amino acids to form “kinks” or to initiate turns to facilitate the “wrapping” of the MSP around the edge of a discoidal phospholipid bilayer. The punctuation region (or sequence) can include from one to 10 amino acids (1, 2, 3, 4, 5, 6, 7, 8, 9 or 10 amino acids), such as 3 to 10 amino acids where there are antiparallel beta sheets in the MSP.

In order to generate smaller belts around the bilayer structure, the overall length of the helical building blocks can be reduced, and the punctuations may be introduced more frequently. The exact amino acid sequence can vary in the positioning and number of the hydrophobic amino acids within the designed linear sequence. Simple models in which either the helical axis is parallel or perpendicular to the normal of the nanodisc bilayer can be generated. To generate a nanodisc with a diameter of roughly 10 nm, an MSP would include about 12 to about 20 (such as 12, 13, 14, 15, 16, 17, 18, 19 or 20) or more repeating units having this generalized amphipathic sequence. In some instances, this protein would be composed of amphipathic alpha helices each with a length of between 14 and 25 amino acids (such as 14, 15, 16, 17, 18, 19, 20, 21, 22, 23 or 24 amino acids), punctuated in the linear sequence by a residue unfavorable for helix formation, such as proline or glycine, or a sequence from about 1 to 5 amino acids (such as 1, 2, 3, 4 or 5 amino acids) which does not favor helix formation, which form small helical building blocks that stabilize the hydrophobic core of the phospholipid bilayer. A helix of about 20-25 amino acids has a height comparable to the thickness of a membrane bilayer. These small helical segments are linked together with from 0 to about 5 amino acid residues (such as 0, 1, 2, 3, 4 or 5), especially glycine or proline.

Table 1 provides sequences of exemplary helical building blocks that can be used to generate artificial MSPs, and further provides a list of specific MSP variants generated using these helical building blocks.

TABLE 1
Helical domain sequences of membrane scaffold proteins

| Abbreviation | Description | Amino acid sequence |
|---|---|---|
| H1 | Helix 1 | LKLLDNWDSVTSTFSKLREQLG (SEQ ID NO: 15) |
| H1Δ(1-11) | Truncated Helix 1 | STFSKLREQLG (SEQ ID NO: 16) |
| H1Δ(1-17) | Truncated Helix 1 | REQLG (SEQ ID NO: 17) |
| H2 | Helix 2 | PVTQEFWDNLEKETEGLRQEMS (SEQ ID NO: 18) |
| H3 | Helix 3 | KDLEEVKAKVQ (SEQ ID NO: 19) |
| H4 | Helix 4 | PYLDDFQKKWQEEMELYRQKVE (SEQ ID NO: 20) |
| H5 | Helix 5 | PLRAELQEGARQKLHELQEKLS (SEQ ID NO: 21) |
| H6 | Helix 6 | PLGEEMRDRARAHVDALRTHLA (SEQ ID NO: 22) |
| H7 | Helix 7 | PYSDELRQRLAARLEALKENGG (SEQ ID NO: 23) |
| H8 | Helix 8 | ARLAEYHAKATEHLSTLSEKAK (SEQ ID NO: 24) |
| H9 | Helix 9 | PALEDLRQGLL (SEQ ID NO: 25) |
| H10 | Helix 10 | PVLESFKVSFLSALEEYTKKLNTQ (SEQ ID NO: 26) |
| FX | Original N-term | MGHHHHHHIEGR (SEQ ID NO: 57) |
| TEV | Modified N-term | MGHHHHHHHDYDIPTTENLYFQG (SEQ ID NO: 58) |
| MSP variant | Name | Composition |
| 1 | MSP1 | FX-H1-H2-H3-H4-H5-H6-H7-H8-H9-H10 |
| 2 | MSP2 | FX-H1-H2-H3-H4-H5-H6-H7-H8-H9-H10-GT-H1-H2-H3- |
| | | H4-H5-H6-H7-H8-H9-H10 |
| 3 | MSP1E1 | FX-H1-H2-H3-H4-H4-H5-H6-H7-H8-H9-H10 |
| 4 | MSP1E2 | FX-H1-H2-H3-H4-H5-H4-H5-H6-H7-H8-H9-H10 |
| 5 | MSP1E3 | FX-H1-H2-H3-H4-H5-H6-H4-H5-H6-H7-H8-H9-H10 |
| 6 | MSP1D1 | TEV-H1Δ(1-11)-H2-H3-H4-H5-H6-H7-H8-H9-H10 |
| 7 | MSP1D2 | TEV-H2-H3-H4-H5-H6-H7-H8-H9-H10 |
| 8 | MSP2N1 | TEV-H1Δ(1-11)-H2-H3-H4-H5-H6-H7-H8-H9-H10-GT- |
| | | H1A(1-11)-H2-H3-H4-H5-H6-H7-H8-H9-H10 |
| 9 | MSP2N2 | TEV-H1Δ(1-11)-H2-H3-H4-H5-H6-H7-H8-H9-H10-GT-H2- |
| | | H3-H4-H5-H6-H7-H8-H9-H10 |
| 10 | MSP2N3 | TEV-H1Δ(1-11)-H2-H3-H4-H5-H6-H7-H8-H9-H10-GT- |
| | | H1Δ(1-17)-H2-H3-H4-H5-H6-H7-H8-H9-H10 |

As noted above, various sizes of ND can be generated, using longer or shorter MSPs and the corresponding number of lipids. The MSP1D1 family results in approximately 10 nm ND and the MSP1E3D1 family forms approximately 12 nm ND. Nanodiscs from about 7 nm to about 17 nm have been formed (Denisov and Sligar, Chem Rev 117(6):4669-4713, 2017). The fundamental discovery underlying the ND technology is that membrane proteins, if added to the detergent solubilized mixture of MSP and phospholipid will self-assemble into the resultant discoidal bilayer. The target protein is in a native-like bilayer environment and displays its native conformation and activity. Membrane proteins of many different topologies, including multi-protein and multi-subunit complexes, also self-assemble in the correct configuration. Oligomeric membrane proteins can be readily incorporated into ND with the correct subunit topology. Examples include the HIV trimeric trans-membrane domain (Reichart et al., Angewandte Chemie 55(8):2688-2692, 2016), dimeric G-Protein coupled receptors (Bayburt et al., J Biol Chem 282(20):14875-14881, 2007), the trimer of dimers of chemotactic receptors (Boldog et al., Proc Natl Acad Sci USA 103(31):11509-11514, 2006), influenza virus hemagglutinin (Bhattacharya et al., J Virol 84(1):361-71, 2010), the tetrameric Trp channel (Gao et al., Nature 534(7607):347-351, 2016) and various transporters and multi-subunit enzymes.

Exemplary MSP sequences that can be used in the immunogenic compositions disclosed herein are provided below (see also U.S. Pat. No. 7,592,008, which is herein incorporated by reference). In some examples, the MSPs include a protein tag, such as a His tag, and optionally further include a protease cleavage site, such as a Factor X (FX) cleavage site or a tobacco etch virus (TEV) protease cleavage site.

MSP1
(SEQ ID NO: 27)
MAKLLDNWDSVTSTFSKLREQLGPVTQEFWDNLEKETEGLRQEMSKDLEEVKAKVQPYL
DDFQKKWQEEMELYRQKVEPLRAELQEGARQKLHELQEKLSPLGEEMRDRARAHVDAL
RTHLAPYSDELRQRLAARLEALKENGGARLAEYHAKATEHLSTLSEK
AKPALEDLRQGLLPVLESFKVSFLSALEEYTKKLNTQ
MSP1 (with His tag/FX)
(SEQ ID NO: 28)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSPLGEEMR
DRARAHVDALRTHLAPYSDELRQRLAARLEALKENGGARLAEYHAKATEHLSTLSEKAK
PALEDLRQGLLPVLESFKVSFLSALEEYTKKLNTQ
MSP2 (with His tag/FX site)
(SEQ ID NO: 29)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSPLGEEMR
DRARAHVDALRTHLAPYSDELRQRLAARLEALKENGGARLAEYHAKATEHLSTLSEKAK
PALEDLRQGLLPVLESFKVSFLSALEEYTKKLNTQGTLKLLDNWDSVTSTFSKLREQLGPV
TQEFWDNLEKETEGLRQEMSKDLEEVKAKVQPYLDDFQKKWQEEMELYRQKVEPLRAE
LQEGARQKLHELQEKLSPLGEEMRDRARAHVDALRTHLAPYSDELRQRLAARLEALKEN
GGARLAEYHAKATEHLSTLSEKAKPALEDLRQGLLPVLESFKVSFLSALEEYTKKLNTQ
MSP1D5D6 (with His tag/FX site)
(SEQ ID NO: 30)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPYSDELRQRLAARLEALKENGGARLAEY
HAKATEHLSTLSEKAKPALEDLRQGLLPVLESFKVSFLSALEEYTKKLNTQ
MSP1D6D7 (with His tag/FX site)
(SEQ ID NO: 31)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSARLAEYH
AKATEHLSTLSEKAKPALEDLRQGLLPVLESFKVSFLSALEEYTKKLNTQ
MSP1D3 (with His tag/FX site)
(SEQ ID NO: 32)
MGHHHHHHIEGRLKLLDNWDSVTSTFSKLREQLGPVTQEFWDNLEKETEGLRQEMSPYL
DDFQKKWQEEMELYRQKVEPLRAELQEGARQKLHELQEKLSPLGEEMRDRARAHVDAL
RTHLAPYSDELRQRLAARLEALKENGGARLAEYHAKATEHLSTLSEKAKPALEDLRQGLL
PVLESFKVSFLSALEEYTKKLNTQ
MSP1D9 (with His tag/FX site)
(SEQ ID NO: 33)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSPLGEEMR
DRARAHVDALRTHLAPYSDELRQRLAARLEALKENGGARLAEYHAKATEHLSTLSEKAK
PVLESFKVSFLSALEEYTKKLNTQ
MSP241 (with His tag/FX site)
(SEQ ID NO: 34)
MGHHHHHHIEGRLKLLDNWDSVTSTFSKLREQLGPVTQEFWDNLEKETEGLRQEMSPYL
DDFQKKWQEEMELYRQKVEPLRAELQEGARQKLHELQEKLSPLGEEMRDRARAHVDAL
RTHLAPYSDELRQRLAARLEALKENGGARLAEYHAKATEHLSTLSEKAKPALEDLRQGLL
PVLESFKVSFLSALEEYTKKLNTQGTLKLLDNWDSVTSTFSKLREQLGPVTQEFWDNLEKE
TEGLRQEMSPYLDDFQKKWQEEMELYRQKVEPLRAELQEGARQKLHELQEKLSPLGEEM
RDRARAHVDALRTHLAPYSDELRQRLAARLEALKENGGARLAEYHAKATEHLSTLSEKA
KPALEDLRQGLLPVLESFKVSFLSALEEYTKKLNTQ
MSP1T4 (with His tag/TEV site)
(SEQ ID NO: 35)
MGHHHHHHHDYDIPTTENLYFQGSVTQEFWDNLEKETEGLRQEMSKDLEEVKAKVQPYL
DDFQKKWQEEMELYRQKVEPLRAELQEGARQKLHELQEKLSPLGEEMRDRARAHVDAL
RTHLAPYSDELRQRLAARLEALKENGGARLAEYHAKATEHLSTLSEKAKPALEDLRQGLL
PVLESFKVSFLSALEEYTKKLNTQ
MSP1T5 (with His tag/TEV site)
(SEQ ID NO: 36)
MGHHHHHHHDYDIPTTENLYFQGKETEGLRQEMSKDLEEVKAKVQPYLDDFQKKWQEE
MELYRQKVEPLRAELQEGARQKLHELQEKLSPLGEEMRDRARAHVDALRTHLAPYSDEL
RQRLAARLEALKENGGARLAEYHAKATEHLSTLSEKAKPALEDLRQGLLPVLESF
KVSFLSALEEYTKKLNTQ
MSP1T6 (with His tag/TEV site)
(SEQ ID NO: 37)
MGHHHHHHHDYDIPTTENLYFQGKDLEEVKAKVQPYLDDFQKKWQEEMELYRQKVEPL
RAELQEGARQKLHELQEKLSPLGEEMRDRARAHVDALRTHLAPYSDELRQRLAARLEAL
KENGGARLAEYHAKATEHLSTLSEKAKPALEDLRQGLLPVLESFKVSFLSALEEY
TKKLNTQ
MSP1N1 (with His tag/TEV site)
(SEQ ID NO: 38)
MGHHHHHHHDYDIPTTENLYFQGSVTQEFWDNLEKETEGLRQEMSKDLEEVKAKVQPYL
DDFQKKWQEEMELYRQKVEPYLDDFQKKWQEEMELYRQKVEPLRAELQEGARQKLHEL
QEKLSPLGEEMRDRARAHVDALRTHLAPYSDELRQRLAARLEALKENGGARLAEYHAKA
TEHLSTLSEKAKPALEDLRQGLL
MSP1E3TEV (with His tag/TEV site)
(SEQ ID NO: 39)
MGHHHHHHHDYDIPTTENLYFQGLKLLDNWDSVTSTFSKLREQLGPVTQEFWDNLEKET
EGLRQEMSKDLEEVKAKVQPYLDDFQKKWQEEMELYRQKVEPLRAELQEGARQKLHEL
QEKLSPLGEEMRDRARAHVDALRTHLAPYLDDFQKKWQEEMELYRQKVEPLRAELQEGA
RQKLHELQEKLSPLGEEMRDRARAHVDALRTHLAPYSDELRQRLAARLEALKENGGARL
AEYHAKATEHLSTLSEKAKPALEDLRQGLLPVLESFKVSFLSALEEYTKKL NTQ
MSP1E3D1 (with His tag/TEV site)
(SEQ ID NO: 40)
MGHHHHHHHDYDIPTTENLYFQGSTFSKLREQLGPVTQEFWDNLEKETEGLRQEMSKDLE
EVKAKVQPYLDDFQKKWQEEMELYRQKVEPLRAELQEGARQKLHELQEKLSPLGEEMRD
RARAHVDALRTHLAPYLDDFQKKWQEEMELYRQKVEPLRAELQEGARQKLHELQEKLSP
LGEEMRDRARAHVDALRTHLAPYSDELRQRLAARLEALKENGGARLAEYHAKATEHLST
LSEKAKPALEDLRQGLLPVLESFKVSFLSALEEYTKKLNTQ
MSP2 (with His tag/TEV site
(SEQ ID NO: 41)
MGHHHHHHHDYDIPTTENLYFQGLKLLDNWDSVTSTFSKLREQLGPVTQEFWDNLEKET
EGLRQEMSKDLEEVKAKVQPYLDDFQKKWQEEMELYRQKVEPLRAELQEGARQKLHEL
QEKLSPLGEEMRDRARAHVDALRTHLAPYSDELRQRLAARLEALKENGGARLAEYHAKA
TEHLSTLSEKAKPALEDLRQGLLPVLESFKVSFLSALEYTKKLNTQGTLKLLDNWDSVTST
FSKLREQLGPVTQEFWDNLEKETEGLRQEMKDLEEVKAKVQPYLDDFQKKWQEEMELYR
QKVEPLRAELQEGARQKLHELQEKLSPLGEEMRDRARAHVDALRTHLAPYSDELRQRLA
ARLEALKENGGARLAEYHAKATEHLSTLSEKAKPALEDLRQGLLPVLESFKVSFLSALEEY
TKKLNTQ
MSP2N1 (with His tag/TEV site)
(SEQ ID NO: 42)
MGHHHHHHHDYDIPTTENLYFQGSTFSKLREQLGPVTQEFWDNLEKETEGLRQEMSKDLE
EVKAKVQPYLDDFQKKWQEEMELYRQKVEPLRAELQEGARQKLHELQEKLSPLGEEMRD
RARAHVDALRTHLAPYSDELRQRLAARLEALKENGGARLAEYHAKATEHLSTLSEKAKP
ALEDLRQGLLPVLESFKVSFLSALEEYTKKLNTQGTFSKLREQLGPVTQEFWDNLEKETEG
LRQEMSKDLEEVKAKVQPYLDDFQKKWQEEMELYRQKVEPLRAELQEGARQKLHELQE
KLSPLGEEMRDRARAHVDALRTHLAPYSDELRQRLAARLEALKENGGARLAEYHAKATE
HLSTLSEKAKPALEDLRQGLLPVLESFKVSFLSALEEYTKKLNTQ
MSP2N2 (with His tag/TEV site)
(SEQ ID NO: 43)
MGHHHHHHHDYDIPTTENLYFQGSTFSKLREQLGPVTQEFWDNLEKETEGLRQEMSKDLE
EVKAKVQPYLDDFQKKWQEEMELYRQKVEPLRAELQEGARQKLHELQEKLSPLGEEMRD
RARAHVDALRTHLAPYSDELRQRLAARLEALKENGGARLAEYHAKATEHLSTLSEKAKP
ALEDLRQGLLPVLESFKVSFLSALEEYTKKLNTQGTPVTQEFWDNLEKETEGLRQEMSKD
LEEVKAKVQPYLDDFQKKWQEEMELYRQKVEPLRAELQEGARQKLHELQEKLSPLGEEM
RDRARAHVDALRTHLAPYSDELRQRLAARLEALKENGGARLAEYHAKATEHLSTLSEKA
KPALEDLRQGLLPVLESFKVSFLSALEEYTKKLNTQ
MSP2N3 (with His tag/TEV site)
(SEQ ID NO: 44)
MGHHHHHHHDYDIPTTENLYFQGSTFSKLREQLGPVTQEFWDNLEKETEGLRQEMSKDLE
EVKAKVQPYLDDFQKKWQEEMELYRQKVEPLRAELQEGARQKLHELQEKLSPLGEEMRD
RARAHVDALRTHLAPYSDELRQRLAARLEALKENGGARLAEYHAKATEHLSTLSEKAKP
ALEDLRQGLLPVLESFKVSFLSALEEYTKKLNTQGTREQLGPVTQEFWDNLEKETEGLRQE
MSKDLEEVKAKVQPYLDDFQKKWQEEMELYRQKVEPLRAELQEGARQKLHELQEKLSPL
GEEMRDRARAHVDALRTHLAPYSDELRQRLAARLEALKENGGARLAEYHAKATEHLSTL
SEKAKPALEDLRQGLLPVLESFKVSFLSALEEYTKKLNTQ
MSP2N4 (with His tag/TEV site)
(SEQ ID NO: 45)
MGHHHHHHHDYDIPTTENLYFQGSVTQEFWDNLEKETEGLRQEMSKDLEEVKAKVQPYL
DDFQKKWQEEMELYRQKVEPLRAELQEGARQKLHELQEKLSPLGEEMRDRARAHVDAL
RTHLAPYSDELRQRLAARLEALKENGGARLAEYHAKATEHLSTLSEKAKPALEDLRQGLL
PVLESFKVSFLSALEEYTKKLNTQNPGTPVTQEFWDNLEKETEGLRQEMSKDLEEVKAKV
QPYLDDFQKKWQEEMELYRQKVEPLRAELQEGARQKLHELQEKLSPLGEEMRDRARAHV
DALRTHLAPYSDELRQRLAARLEALKENGGARLAEYHAKATEHLSTLSEKAKPALEDLRQ
GLLPVLESFKVSFLSALEEYTKKLNTQ
MSP2N5 (with His tag/TEV site)
(SEQ ID NO: 46)
MGHHHHHHHDYDIPTTENLYFQGSVTQEFWDNLEKETEGLRQEMSKDLEEVKAKVQPYL
DDFQKKWQEEMELYRQKVEPYLDDFQKKWQEEMELYRQKVEPLRAELQEGARQKLHEL
QEKLSPLGEEMRDRARAHVDALRTHLAPYSDELRQRLAARLEALKENGGARLAEYHAKA
TEHLSTLSEKAKPALEDLRQGLLNPGTKDLEEVKAKVQPYLDDFQKKWQEEMELYRQKV
EPYLDDFQKKWQEEMELYRQKVEPLRAELQEGARQKLHELQEKLSPLGEEMRDRARAHV
DALRTHLAPYSDELRQRLAARLEALKENGGARLAEYHAKATEHLSTLSEKAKPALEDLRQ
GLLPVTQEFWDNLEKETEGLRQEMS
MSP2N6 (with His tag/TEV site)
(SEQ ID NO: 47)
MGHHHHHHHDYDIPTTENLYFQGSVTQEFWDNLEKETEGLRQEMSKDLEEVKAKVQPYL
DDFQKKWQEEMELYRQKVEPYLDDFQKKWQEEMELYRQKVEPLRAELQEGARQKLHEL
QEKLSPLGEEMRDRARAHVDALRTHLAPYSDELRQRLAARLEALKENGGARLAEYHAKA
TEHLSTLSEKAKPALEDLRQGLLSNPGTQKDLEEVKAKVQPYLDDFQKKWQEEMELYRQ
KVEPYLDDFQKKWQEEMELYRQKVEPLRAELQEGARQKLHELQEKLSPLGEEMRDRARA
HVDALRTHLAPYSDELRQRLAARLEALKENGGARLAEYHAKATEHLSTLSEKAKPALEDL
RQGLLPVTQEFWDNLEKETEGLRQEMS
MSP2CPR (with His tag/FX site)
(SEQ ID NO: 48)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSPLGEEMR
DRARAHVDALRTHLAPYSDELRQRLAARLEALKENGGARLAEYHAKATEHLSTLSEKAK
PALEDLRQGLLPVLESFKVSFLSALEEYTKKLNTQGTLKLLDNWDSVTSTFSKLREQLGPV
TQEFWDNLEKETEGLRQEMSKDLEEVKAKVQPYLDDFQKKWQEEMELYRQKVEPLRAE
LQEGARQKLHELQEKLSPLGEEMRDRARAHVDALRTHLAPYSDELRQRLAARLEALKEN
GGARLAEYHAKATEHLSTLSEKAKPALEDLRQGLLPVLESFKVSFLSALEEYTKKLNTQST
MGDSHEDTSATMPEAVAEEVSLFSTTDMVLFSLIVGVLTYWFIFRKKKEEIPEFSKIQTTAP
PVKESSFVEKMKKTGRNIIVFYGSQTGTAEEFANRLSKDAHRYGMRGMSADPEEYDLADL
SSLPEIDKSLVVFCMATYGEGDPTDNAQDFYDWLQETDVDLTGVKFAVFGLGNKTYEHF
NAMGKYVDQRLEQLGAQRIFELGLGDDDGNLEEDFITWREQFWPAVCEFFGVEATGEESS
IRQYELVVHEDMDVAKVYTGEMGRLKSYENQKPPFDAKNPFLAAVTANRKLNQGTERHL
MHLELDISDSKIRYESGDHVAVYPANDSALVNQIGEILGADLDVIMSLNNLDEESNKKHPF
PCPTTYRTALTYYLDITNPPRTNVLYELAQYASEPSEQEHLHKMASSSGEGKELYLSWVVE
ARRHILAILQDYPSLRPPIDHLCELLPRLQARYYSIASSSKVHPNSVHICAVAVEYEAKSGRV
NKGVATSWLRAKEPAGENGGRALVPMFVRKSQFRLPFKSTTPVIMVGPGTGIAPFMGFIQE
RAWLREQGKEVGETLLYYGCRRSDEDYLYREELARFHKDGALTQLNVAFSREQAHKVYV
QHLLKRDREHLWKLIHEGGAHIYVCGDARNMAKDVQNTFYDIVAEFGPMEHTQAVDYV
KKLMTKGRYSLDVWS
His-TEV-MSP1T2-GT
(SEQ ID NO: 49)
MGHHHHHHHDYDIPTTENLYFQGSTFSKLREQLGPVTQEFWDNLEKETEGLRQEMSKDLE
EVKAKVQPYLDDFQKKWQEEMELYRQKVEPLRAELQEGARQKLHELQEKLSPLGEEMRD
RARAHVDALRTHLAPYSDELRQRLAARLEALKENGGARLAEYHAKATEHLSTLSEKAKP
ALEDLRQGLLPVLESFKVSFLSALEEYTKKLNTQGT
MSP1RC12′ (with His tag/FX site)
(SEQ ID NO: 50)
MGHHHHHHIEGCLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSPLGEEMR
DRARAHVDALRTHLAPYSDELRQRLAARLEALKENGGARLAEYHAKATEHLSTLSEKAK
PALEDLRQGLLPVLESFKVSFLSALEEYTKKLNTQ
MSP1K90C (with His tag/FX site)
(SEQ ID NO: 51)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQCLHELQEKLSPLGEEMR
DRARAHVDALRTHLAPYSDELRQRLAARLEALKENGGARLAEYHAKATEHLSTLSEKAK
PALEDLRQGLLPVLESFKVSFLSALEEYTKKLNTQ
MSP1K152C (with His tag/FX site)
(SEQ ID NO: 52)
MGHHHHHHIEGRLKLLDNWDSVTSTFSKLREQLGPVTQEFWDNLEKETEGLRQEMSKDL
EEVKAKVQPYLDDFQKKWQEEMELYRQKVEPLRAELQEGARQKLHELQEKLSPLGEEMR
DRARAHVDALRTHLAPYSDELRQRLAARLEALKENGGARLAEYHACATEHLSTLSEKAK
PALEDLRQGLLPVLESFKVSFLSALEEYTKKLNTQ

### VI. Phospholipids

The immunogenic compositions disclosed herein include at least one phospholipid. Phospholipids are a type of lipid molecule that include a phosphate group joined to one or more (such as two) hydrocarbon tails, which are typically long fatty acid chains that may be saturated or unsaturated. The phosphate group may further be joined to any suitable substituent group such as a choline, ethanolamine, glycerol, inositol, or serine.

In some implementations herein, the phospholipid is a glycerophospholipid, an ether glycerophospholipid, or a sphingophospholipid. Glycerophospholipids are phospholipids that have a glycerol group between the phosphate and the hydrocarbon tails (e.g., the long fatty acid chains that may be saturated or unsaturated). Ether glycerophospholipids are phospholipids in which one or more of the carbons on the glycerol group are joined to a hydrocarbon tail via an ether linkage. Sphingophopholipids are a type of phospholipid having a backbone of sphingoid bases. Phospholipids are amphipathic-having both a polar/hydrophilic portion and a neutral/hydrophobic portion.

The selected phospholipid(s) can contain glycerol backbones, sphingolipids or ether linkages. Generally, the phospholipid has two saturated or partially unsaturated fatty acids of from 6 to 20 carbon atoms (such as 6, 7, 8, 9, 10, 11, 12, 13, 14, 15, 16, 17, 18, 19 or 20) with a commonly used head group such as, but not limited to, phosphatidyl choline, phosphatidyl ethanolamine and phosphatidyl serine. The head group can be uncharged, positively charged, negatively charged or zwitterionic. The phospholipids can be natural or synthetic, or mixtures of natural and synthetic. In some implementations, the molar ratio of MSP to total membrane protein is that which produces about 100 to about 200 phospholipid molecules (such as about 100, about 125, about 150, about 175 or about 200 phospholipid molecules) in each discoidal structure of about 10 nm in diameter, such as about 8 to about 12 nm or about 9 to about 11 nm in diameter. Those proteins, found in nature or associated with the various membrane structures of a living organism, are solubilized in the MSP supported ND through the process of self-assembly, and the native structure and activity of the target protein are preserved in these MSP-supported structures.

In some implementations of the present disclosure, the glycerophospholipid includes phosphatidyl choline, phosphatidyl ethanolamine, phosphatidyl serine, phosphatidyl inositol, cardiolipin, lysophospholipid, dipalmitoyl-phosphatidylcholine, dimyristoyl phosphatidyl choline, 1-palmitoyl-2-oleoyl-phosphatidyl choline, 1-palmitoyl-2-oleoyl-phosphatidyl serine, 1-palmitoyl-2-oleoyl-phosphatidyl ethanolamine, dioleoylphosphoethanolamine, dioleoylphosphatidylcholine, dihexanoyl phosphatidyl choline, dipalmitoyl phosphatidyl ethanolamine, dipalmitoyl phosphatidyl inositol, dimyristoyl phosphatidyl ethanolamine, dimyristoyl phosphatidyl inositol, dihexanoyl phosphatidyl ethanolamine, dihexanoyl phosphatidyl inositol, 1-palmitoyl-2-oleoyl-phosphatidyl ethanolamine, 1-palmitoyl-2-oleoyl-phosphatidyl inositol, or any combination thereof.

In some implementations, the ether glycerophospholipid includes 1,2-di-O-phytanyl-sn-glycero-3-phosphocholine, 1,2-di-O-phytanyl-sn-glycero-3-phosphoethanolamine, 1,2-di-O-phytanyl-sn-glycerol, glycerol dialkyl glycerol tetraether, 1,2-di-O-octadecyl-sn-glycero-3-phosphocholine, 1,2-di-O-(9Z-octadecenyl)-sn-glycero-3-phosphocholine, 2-3-diphytanyl-O-sn-glycerol, caldarcheol, isocalarcheol, gentiobiosyl archaeol, archaetidylethanoloamine, gentyobiosyl caldarc haetidylethanoloamine, or any combination thereof.

In some implementations, the sphingophospholipid comprises sphingomyelin.

The following examples are provided to illustrate certain particular features and/or implementations. These examples should not be construed to limit the disclosure to the particular features or implementations described.

### EXAMPLES

### Example 1: Production and Characterization of Nanodiscs Containing Influenza Virus M2 (M2:ND)

This example describes the manufacture of NDs expressing M2 (M2:ND) and assessment of the immunogenicity of these assemblies.

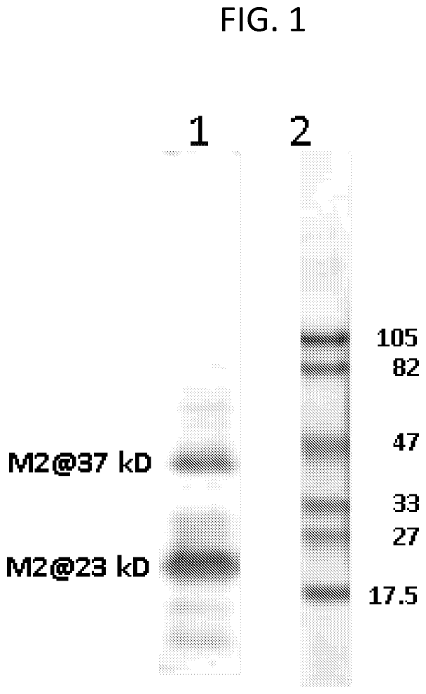

A process to assemble ND expressing recombinant M2 produced in E. coli, under conditions that allow M2 oligomerization, was developed. M2 with an N-terminal His tag (SEQ ID NO: 14) was produced in E. coli, purified, and used to manufacture M2:ND using MSP1D1 (see Table 1) as the scaffold protein and DMPC as the lipid (see Example 2). Analysis of the M2:ND by Western blot revealed the presence of two bands that reacted with anti-His-tag monoclonal antibody (FIG. 1). Mass-spectrometry analysis of these two bands confirmed the presence of M2. The 23 kD band represents an M2 monomer, while the 37 kD band represents an M2 oligomer (FIG. 1).

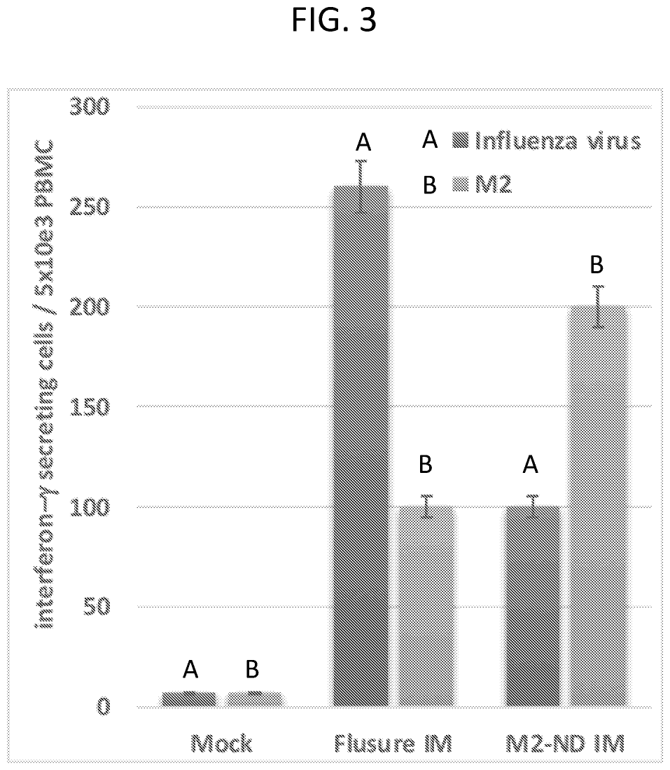

The immunogenicity of the M2:ND assemblies was examined by immunizing a first group of swine intramuscularly (IM) with 20 μg of M2 displayed in ND. For immunization, the M2:ND were mixed with a commercial water:oil:water (w:o:w) adjuvant (MONTANIDE™ ISA 206, Seppic). A second group of swine was IM administered FLUSURE XP®, a commercial tetravalent swine influenza WIV vaccine. Control animals were mock-vaccinated. As shown in FIG. 2, the M2:ND vaccine was able to elicit the production of serum IgG which reacted not only with recombinant M2, but also with influenza A virions. Immunization with FLUSURE XP® led to the production of serum IgG that reacted with influenza A virions, but not recombinant M2 or M2e. As shown in FIG. 3, the M2:ND assemblies also elicited the development of M2-specific T cells. These data demonstrate that NDs displaying M2 (M2:ND) are immunogenic.

### Example 2: Assembly of the M2:ND Vaccine

The external surface of a microbe (such as a virus) displays antigenic proteins that can be targeted by the immune system resulting in efficient antibody production. Surface proteins are imbedded in the membrane, often as complex oligomers that are recalcitrant to simple approaches wherein they are isolated from the membrane with detergents and then purified to homogeneity. Even when membrane proteins have been solubilized by adding detergent or exogenous lipids, their conformations are usually modified and structurally uncertain. These detergent solubilized proteins are usually inactive and do not faithfully represent the structure of antigenic sites that would be found on either side of the bilayer. In the case of the IAV M2 envelope protein, an attempt was made to isolate the extracellular domain of a membrane protein target of M2, the so-called ectodomain (M2e), and then use this for immunization. However, this artificially solubilized piece of the membrane protein did not elicit a strong antibody response. Numerous subsequent attempts have been made to conjugate M2e with various adjuvants, with somewhat improved results; however, these approaches are inherently flawed as they do not address the fundamental issue of having the correct conformation or correct oligomeric structure of the membrane protein antigenic site so as to elicit the correct antibody response.

There have been many attempts to surmount the difficulty of isolating integral membrane proteins in an active state. These include incorporation into liposomes or micellar detergent aggregates. While successful for some in vitro studies, liposomes are inherently unstable and prone to fusion. Placing a single membrane protein target into a ˜100 nm liposome is a challenge and overloads an assay with excess lipids. Liposomal delivery systems, while in some cases applicable for small molecule therapeutics, have generally been unsatisfactory as the basis for vaccines. Proteins solubilized in micelles most often have an altered activity profile. The studies disclosed herein use small lipoprotein stabilized particles termed ND. As noted, the general problem in revealing the structure and function of membrane proteins is that removing them from their lipid bilayer environment results in inactivation, unfolding or critical modifications of activity. Membrane proteins require the presence of a phospholipid bilayer to maintain their functional structure.

To solve these solubility problems, the nanotechnology-based ND (Denisov and Sligar, Chem Rev 117(6):4669-4713, 2017; Bayburt et al., Nano Lett 2(8):853-856, 2002) method was employed. The ND system readily self-assembles a membrane protein into a nanometer scale phospholipid bilayer, with a precisely controlled size from ˜10 nm to ˜17 nm in diameter. The entire entity is stabilized by two encircling amphipathic helical protein belts termed membrane scaffold proteins (MSP). Following self-assembly into ND, the membrane protein is in a native-like environment and displays full functionality, and the entire entity is soluble in aqueous media. The stabilized phospholipid bilayer epitaxially presents both the cytoplasmic and extracellular part of the target for efficient recognition by the immune system. The ND can be assembled with a wide range of synthetic and natural lipids and lipid mixtures. This allows facile co-incorporation of lipid adjuvants such as monophosphorylated lipid A (MLPA) and anchored oligonucleotides.

### Generation of Membrane Scaffold Proteins (MSP) for Vaccine Use

One component in the formation of nanodiscs is the encircling membrane scaffold protein (MSP). Originally motivated by the human Apo-AI protein that is the main polypeptide component of high density lipoproteins (HDL), the sequence has been optimized in order to form the most stable and robust discoidal bilayers (Denisov et al., J Am Chem Soc 126(11):3477-3487, 2004). ND of varying sizes can be used by inserting or deleting additional amphipathic helices into the MSP sequence (see, e.g., U.S. Pat. No. 7,592,008). In some examples herein, the MSP is derived from the porcine Apo-AI sequence, which readily self-assembles to produce monodisperse and homogeneous ND. High level expression of the MSPs in E. coli typically yields 500 mg-1000 mg protein per liter of culture.

### Production of Pyrogen Free MSPs

The studies described herein include the production of highly purified membrane scaffold proteins that are completely free of any endotoxins that are carried over from the bacterial expression. Additional purification steps are used to reduce and eliminate the endotoxin level of the protein preparation. Production of pyrogen free MSP has been described previously, such as in Numata et al. (Int J Nanomedicine 8:1417-1427, 2013). Typically, MSP preparations (100-150 mg) are adsorbed onto a 1 cm×5 cm Ni-NTA column and washed with 10 bed volumes of an aseptically prepared 1% Triton X-100 solution in phosphate buffered saline (PBS), followed by a 10-bed volume wash of buffer without detergent. The MSP is then eluted with a 0.3M imidazole solution and the buffer exchanged using an Amicon 15 centrifugal ultrafiltration concentrator. The sample is filter-sterilized and the endotoxin level is determined using a PYROGENT-5000 assay (Lonza, Walkersville, MD, USA).

### Production and Purification of M2

Greater than 98% of swine IAV isolated in the United States carry the M gene segment derived from the 2009 H1N1 human pandemic (pdm) lineage. Thus, the M2 isoform incorporated into the nanodiscs is a consensus sequence of the M2 H1N1pdm2009 isoform. The consensus sequence was generated based on the analysis of 3816 sequences of IAV isolated from swine in North America from 2012 to 2018, which are available in the Influenza Research Database (Zhang et al., Nucleic Acids Res 45(D1):D466-D74, 2017). An N-terminal poly-histidine tag was added to simplify purification and detection of the target protein. The amino acid sequences of the different segments of the M2 isoform are:

His Tag:
(SEQ ID NO: 9)
MHHHHHHSSGVDLGTENLYFQSNA
N-terminal Domain:
(SEQ ID NO: 10)
MSLLTEVETPTRSEWECRCSDS
TM Helix:
(SEQ ID NO: 11)
SDPLAIAANIIGILHLILWITDRL
Amphipathic Helix:
(SEQ ID NO: 12)
FFKCIYRRFKYGLKRG
C-terminal Domain:
(SEQ ID NO: 13)
PSTEGVPESMREEYQQEQQSAVDVDDGHFVNIELE.
Full sequence of the M2 isoform without the His
tag:
(SEQ ID NO: 1)
MSLLTEVETPTRSEWECRCSDSSDPLAIAANIIGILHLILWITDRLFFK
CIYRRFKYGLKRGPSTEGVPESMREEYQQEQQSAVDVDDGHFVNIELE
Full sequence of the M2 isoform with the His tag:
(SEQ ID NO: 14)
MHHHHHHSSGVDLGTENLYFQSNAMSLLTEVETPTRSEWECRCSDSSDP
LAIAANIIGILHLILWITDRLFFKCIYRRFKYGLKRGPSTEGVPESMRE
EYQQEQQSAVDVDDGHFVNIELE

The synthetic gene of M2 was expressed in E. coli BL21gold (DE3), under the control of the T7 promoter (pET expression system). Expression was optimized with respect to several parameters (e.g., media composition, growth temperature, post-induction time). The best result was achieved using TB medium, a 25° C. post-induction temperature, and collection of cells 18-22 hours after induction. A batch of M2 was produced from the cell pellet of a 4.8 L culture that was resuspended in 400 ml PBS supplemented with 3% Empigen BB, sonicated, and solubilized for 1 hour at 4° C. The lysate was clarified by centrifugation at 35,000 RPM (Ti-45 rotor). Solubilized material was mixed with 5 ml Ni-NTA resin (10 ml of 50% slurry) incubated 1 hour on ice, then the resin was transferred to the chromatography column, washed with buffer containing 20 mM imidazole and detergent (3 different detergents were tested—0.3% Empigen (A), 0.1% DDM (B), or 0.5% octoglucoside (C)), eluted with 0.5 M imidazole and corresponding detergent. M2 co-purified with several E. coli proteins. The co-purified proteins are likely stress-response proteins, which often serve as chaperons.

### Assembly of M2 Protein Into ND

The methodologies and detailed protocols for generating empty ND as well as incorporating membrane proteins into these bilayer nanostructures has been previously described (Ritchie et al., Methods Enzymol 464:211-231, 2009; Luthra et al., Methods Mol Biol 987:115-127, 2013; Denisov and Sligar, Nat Struct Mol Biol 23(6):481-486, 2016; and U.S. Pat. No. 7,592,008). In assembling “bare” ND (those without incorporated protein) the results are assayed by size exclusion chromatography. This analytical technique can readily reveal the generation of a homogenous ND peak verses aggregates of lipid or the membrane scaffold protein. The assembly process involves detergent solubilizing the MSP together with synthetic phospholipid or a mixture of lipid types. The studies disclosed herein use as a bulk lipid in the ND either a saturated lipid (DMPC) or a lipid with a single unsaturated side chain (DOPC). In assembling bare ND, a detergent such as cholate solubilizes both the MSP and the phospholipid. The assembly process is optimized at a temperature near the phase transition temperature of lipids being used. For DMPC, work is done at room temperature. For unsaturated lipids, such as DOPC or POPC, with a bulk phase transition temperature below zero centigrade, work is performed on ice at 4° C.

Incorporation of M2 into NDs was achieved by mixing M2 samples (eluates from Ni-NTA-column) with ND reconstitution mixture [MSP1D1(-):DMPC:cholate molar ratios 1:75:150]. Batches of M2:ND were assembled using a large excess of the reconstitution mixture (which allowed for use of the same lipid to MSP ratio as in empty NDs) and MSP that had the His-tag removed to facilitate the removal of excess of empty ND. After a short incubation, the detergent was removed with amberlite XAD-2 resin. Then the samples were filtered, loaded onto Ni-NTA column, washed with buffer containing 20 mM imidazole, without detergent, and eluted with buffer containing 0.5 M imidazole. All 3 samples looked similar on SDS-PAGE/Western blot: most M2 bound the resin, excess MSP was removed during an extensive washing step, and some MSP was present in the eluates, which is consistent with formation of ND. The samples were analyzed using size-exclusion chromatography followed by SDS-PAGE/Western blot analysis of the fractions. Typically, when membrane protein is getting successfully incorporated into ND formed by MSP1 (10 nm diameter), the hydrodynamic diameter of such complexes falls within a 10-14 nm range, depending on the size, shape and oligomeric state of the target protein. When the incorporation fails, a large peak corresponding to the column void volume is usually observed. The final sample (combined chromatography fractions) corresponding to size 10.5-14 nm, containing both M2 and MSP, was stable in the absence of detergent for a prolonged period of time, and can be diluted, concentrated, subjected to several freeze-thaw cycles without loss of the material. This is in contrast to non-incorporated M2, which in the absence of detergent deteriorated within a day.

### Example 3: Immunization of Swine With M2:ND or a WIV Vaccine With or Without M2:ND Supplementation

This example describes a study to evaluate the protective immunity against swine influenza virus conferred by the immunization of swine with M2:ND, as compared to that afforded by a WIV administered either alone or in combination with M2:ND.

Assessment of the Ability of M2:ND to Provide Protective Immunity and/or Increase the Breadth of Coverage Afforded by Influenza WIV Vaccines Against Antigenically Distinct IAV

Viruses belonging to the swine IAV (swIAV) H3N2 subtype, which is endemic in North America, were selected as the IAV experimental system to test the breadth of protective immunity afforded by WIV vaccines. The H3 phylogenetic clade IV (C-IV) emerged in North America in the mid-2000s as an evolutionary branch of clade III TRIG viruses introduced in the 1990s and has continued to increase in genetic and antigenic diversity. Antigenic cartography studies on the HA of contemporary C-IV H3N2 swIAV, which includes eight monophyletic genetic clades (clades IV, IV-A, IV-B, IV-C, IV, D, IV-E, IV-F, and human like H3), identified several distinct antigenic clusters, with three of them (cyan, green and red antigenic clusters) among the most frequently detected (Abente et al., J Virol 90(18):8266-8280, 2016; Bolton et al., Influenza Other Respir Viruses 13(1):83-90, 2018). These antigenic clusters are the result of a few amino acid changes in the head of the HA H3, which affect the susceptibility of these viruses to hemagglutination inhibition. Based on the predicted substantial diversity of antigenic phenotypes present in the HA of H3N2 viruses circulating in the swine population, it is not expected that a single representative of any of the several C-IV clades of H3N2 swIAV included in a WIV vaccine would protect against all of the antigenically diverse viruses belonging to the various C-IV H3N2 clades. Indeed, it has been shown experimentally that a few amino acid differences in H3 antigenic clusters affect the efficacy of WIV vaccines (Abente et al., J Virol 9(22):e01323-18, 2018). Hence, this system was chosen to examine the breadth of protection afforded by a WIV vaccine with or without M2 supplementation.

### Experimental Design

The efficacy of WIV vaccines was evaluated with or without M2:ND supplementation, prepared using representative virus belonging to one of the C-IV clades of H3N2 swIAV expressing a particular H3 antigenic cluster, against a challenge virus from a different C-IV clade, expressing a disparate H3 antigenic cluster. Supplementation of the WIV influenza vaccine formulation with M2:ND tests the ability of M2:ND to extend the limited breadth of protective immunity afforded by conventional WIV against a heterologous challenge virus exhibiting an antigenically distinct H3 HA molecule. In addition, to explore the efficacy of the M2:NDs as a stand-alone biologic, groups of animals vaccinated only with M2:NDs were also included in the trial.

Genetically distinct representative viruses are selected among members of the C-IV clades of H3N2 swIAV. Pairs of viruses, from different C-IV clades, displaying either the red, green or cyan H3 antigenic groups, are selected as either the vaccine or the challenge virus based on having disparate H3 antigenic clusters. All of the viruses used in these experiments express the pdm isoform of the matrix gene, and the M2:NDs are made using recombinant M2 based on the consensus sequence of the M2 pdm isoform (SEQ ID NO: 1). Thus, the M2:ND immunogen and the challenge virus express the same M2 isoform. As a control for maximum vaccine efficacy, groups of vaccinated and non-vaccinated animals were challenged with the same strain of virus used to prepare the WIV vaccine. The experimental groups are listed in Table 2.

An initial pair of IAV strains that belong to two different C-IV clades of H3N2 swIAV (clade IV-A and clade IV-B) were selected. These two viruses also express distinct H3 molecules which belong, respectively, to the H3 antigenic groups red and green. These viruses were identified by searching the influenza virus database, using as a primary parameter for their selection the article by Bolton et al. (Influenza Other Respir Viruses 13(1):83-90, 2018), which describes the antigenic distances between different H3 antigenic clusters. The goal of the selection was to obtain a virus combination in which vaccination with a WIV vaccine prepared from one of them would only be expected to provide partial protection against the other. The virus selected as the vaccine virus is: Clade IV-A, A/swine/NY/A01104005/2011 (H3N2) (NY11), “red antigenic motif” H3N2 (NYNNYK; SEQ ID NO: 53). The virus selected as the challenge virus is: Clade IV-B, A/swine/Minnesota/A01668936/2016 (H3N2) (MN16), “green antigenic motif” H3N2 (KYNNYK; SEQ ID NO: 54). Both viruses are derived from the same 1998 introduction/lineage and both have the same pdm-Matrix gene and therefore express an identical M2 isoform. Genome pattern for both viruses is 2002-lineage NA, T-T-T-T-P-T.

### Animal Studies and Measurement of Protective Immunity

The efficacy of WIV vaccines, with or without M2:ND supplementation, was determined by measuring viral load in the lung lavage fluids (LLF). Evidence of protection is defined as statistically significant reduction in virus titers in LLF compared to the non-vaccinated groups. The list of treatment groups in the vaccination and challenge experiment is shown in Table 2. Three-week old pigs were treated with ceftiofur crystalline antibiotic (EXCEDE™, Pharmacia & Upjohn Company) per manufacturer's recommended dose and randomly allocated into the groups listed in Table 1. Each treatment group was housed separately in pens at a suite of a BSL2 containment facility for 1 week prior to immunization. Although due to the influence of sex hormones, the response to vaccines by females tends to be higher (Giefing-Kroll et al., Aging Cell 14(3):309-321, 2015), since young pigs are being used several months before they reach puberty, it is not expected that gender will influence the results of these experiments. All pigs were confirmed seronegative for swine influenza virus by hemagglutination inhibition assays against influenza virus and were free of influenza virus infection by virus isolation from nasal swabs collected on the day of vaccination. The WIV vaccine was prepared as previously described (Lager and Vincent, Methods Mol Biol 1161:355-361, 2014; Loving et al., J Virol 87(17):9895-9903, 2013) using a dose of 320 HA units of UV-irradiated virus adjuvanted with the commercial o:w adjuvant EMULSIGEN®-D (MVP Adjuvants) in a 2 ml volume. The supplementation of the WIV vaccine with M2:ND was done by adding into the suspension of the inactivated virus 20 μg of M2 displayed in the M2:ND and then mixed with the w:o:w adjuvant MONTANIDE™ ISA 206 (Seppic) in a 2 ml volume.

The vaccine was administered by intramuscular injection twice at a 3-week interval. Fourteen days after the second vaccination, the animals were challenged intratracheally with 105 tissue culture infectious dose 50 (TCID50)/ml of the selected challenge virus. A group of strict controls that were not vaccinated or challenged was included in the trial. All pigs enrolled in the study were humanely euthanized at 5 days after being challenged and their lungs examined for evidence of macroscopic pneumonia, and LLF were collected thereafter. Pigs were observed daily for clinical signs during the 5 days after challenge. Serum samples were collected 0, 21, and 35-days post vaccination. Anticoagulated whole blood for isolation of mononuclear cells to assess cell mediated immunity (CMI) are collected 0, 21 and 35-days post vaccination. Pigs were euthanized and necropsied 5 days post challenge. LLF was collected for virus titration as described previously, using 50 ml Minimum Essential Medium per lung lavage (Larsen et al., Vet Microbiol 74(1-2):117-131, 2000; Vincent et al., Vaccine 25(47):7999-8009, 2007). Viral titers were determined using a plaque forming unit assay using MDCK cell monolayers as the substrate for virus replication (Landreth et al., Vet Microbiol 253:108968, 2021). The presence of macroscopic lung lesions for all seven lung lobes was examined and scored as previously described (Halbur et al., Vet Pathol 32(6):648-660, 1995).

TABLE 2
Animal study design

| | Vaccine Virus* | | Challenge virus* | | | |
|---|---|---|---|---|---|---|
| | H3N2 | H3 | H3N2 | H3 | | Expected |
| Experimental | genetic clade | antigenic | genetic clade | antigenic | Pigs per | protection |
| group | (strain name) | cluster | (strain name) | cluster | group | level |
| WIV | IV-A (NY11) | Red | IV-A (NY11) | Red | 5 | Complete |
| Mock vaccine | — | — | IV-A (NY11) | Red | 4 | None |
| WIV | IV-A (NY11) | Red | IV-B (MN16) | Green | 8 | Partial |
| M2:ND | — | — | IV-B (MN16) | Green | 8 | |
| WIV + | IV-A (NY11) | Red | IV-B (MN16) | Green | 8 | |
| M2:ND | | | | | | |
| Mock vaccine | — | — | IV-B (MN16) | Green | 4 | None |
| Strict control | — | — | — | — | 4 | NA |

*The selected H3N2 strain to produce the WIV vaccine expressing the Red antigenic cluster is NY11-[A/swine/NY/A01104005/2011(H3N2)]. The H3N2 strain selected as the challenge virus expressing the Green antigenic cluster is MN16 - [A/swine/Minnesota/A01668936/2016(H3N2)].

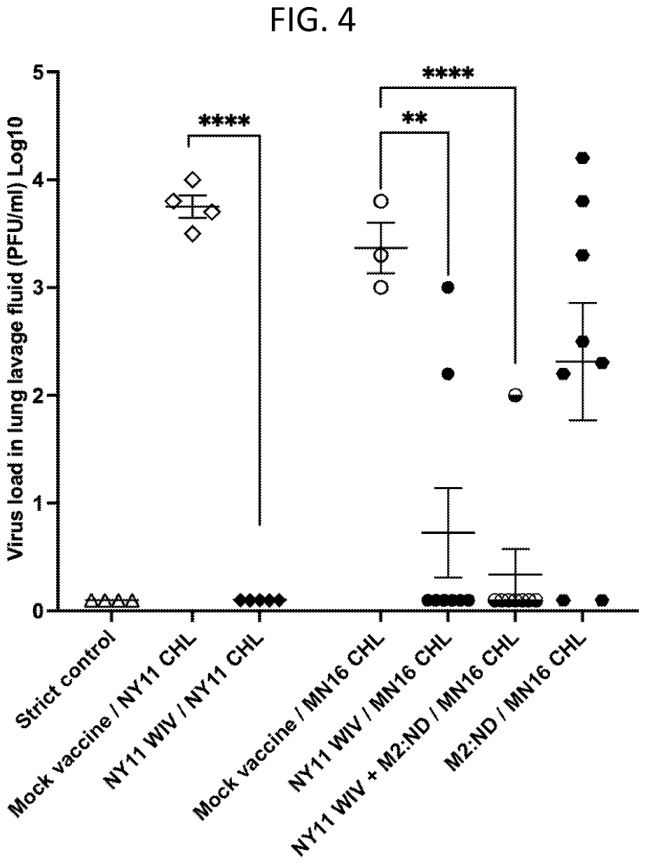

As shown in FIG. 4, the group of pigs vaccinated with the NY11 based WIV vaccine and challenged with the NY11 (filled diamonds in FIG. 4) exhibited complete protection from virus challenge, as indicated by the absence of infectious virus in the LLF of all the pigs in this group. In contrast, all the mock-vaccinated pigs challenged with the NY11 virus (clear diamonds in FIG. 4) exhibited a significant amount of infectious virus in their LLF. None of the pigs in the strict control group (clear triangles in FIG. 4) had detectable virus in their LLF. On the other hand, two of the eight pigs in the group immunized with the NY11 based WIV vaccine and challenged with the MN16 virus (filled circles in FIG. 4) had infectious virus present in their LLF, indicating partial protection of this group against the antigenically distinct virus challenge. The level of protective immunity provided by the NY11-based WIV vaccine against the challenge with MN16 virus was slightly enhanced by the addition of the M2:ND in the vaccine (half-filled circles in FIG. 4). Notably, as compared to the mock-vaccinated and MN16-challenged group, the supplementation of the WIV vaccine with M2:ND resulted in a higher level of statistical significance (P<0.0001) than that attained with the non-supplemented vaccine (P<0.01). Remarkably, a biologically favorable result indicating vaccine efficacy was observed in the group immunized with the M2:ND vaccine (filled hexagons in FIG. 4). This is indicated by the absence of infectious virus in two of the eight pigs in this group, as well as a lower virus load in the LLF of three of the pigs, as compared to the mock vaccinated pigs challenged with the MN16 virus (open circles in FIG. 4). One of the pigs in this group died from causes unrelated to the virus challenge.

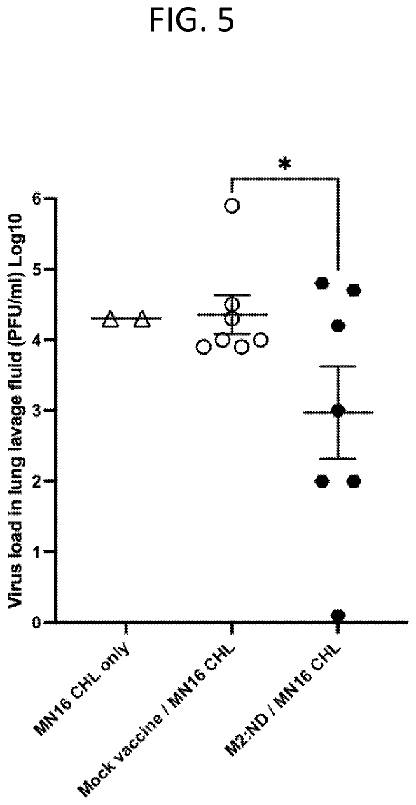

An additional experiment was conducted to test the efficacy of the M2:ND as a stand-alone biologic using a larger number of mock-vaccinated pigs for comparison. As shown in FIG. 5, the mean lung virus load in the group of pigs immunized with the M2:NDs and challenged with the MN16 virus (filled hexagons in FIG. 5) exhibited a significantly lower virus load (P<0.05) as compared to the mock vaccinated group (clear circles in FIG. 5). In both experiments, the extent of gross lung pathology resulting from the virus challenge was highly variable and did not correlate with the presence of virus load in the LLF. Hence, it was not used in the analysis of vaccine efficacy. Combined, the evidence presented demonstrates that the M2:ND vaccine is a valuable biologic for broadening the protective efficacy of type A influenza vaccines.

### Example 4: Optimization of M2:ND Construction and Assessment of the Oligomerization State of M2 in the ND Assemblies

This example describes studies to determine the optimal amount of phospholipid for the M2:ND composition.

Different phospholipid ratios and size exclusion chromatography are used to assess M2:ND formation. Titration experiments are performed for each phospholipid used to optimize the incorporation of full-length M2 that favor the formation of M2 tetramers into NDs. Incorporating membrane proteins into the lipid bilayer of a ND involves a process of self-assembly. Detergent solubilizes the target phospholipids and the membrane scaffold protein. The gradual removal of detergent allows partners to find each other, the target protein with boundary lipids, bulk lipids forming a bilayer and the entire nanometer scale discoidal particle stabilized by the encircling membrane scaffold protein. If the target protein tends to oligomerize, as is the case of the tetrameric M2, then these protein-protein interfaces form prior to lipid assembly. For high affinity interactions, the oligomer is functional, with all subunit monomers organized in the correct configuration. For incorporation of M2, ratios of scaffold protein, M2 and lipids are selected to favor tetramer formation. Following verification of efficacy in the animal model, the oligomeric state of M2 in the ND is evaluated by separation of various M2 in discs by flotation on sucrose gradient. The different sizes and/or densities of ND with varying M2 and lipid are distinguished as described (Denisov and Sligar, Chem Rev 117:4669-4713, 2017; Ginkova et al., Biochem Biophys Res Comm 398:194-198, 2010; Bayburt et al., J Biol Chem 282:14875-14881, 2007). Multi-angle light scattering coupled with size exclusion chromatography, which allows determination of particle mass, is also used. Active M2:ND preparations are analyzed with mass spectrometry. It has been demonstrated that incorporated target mass can be determined (Marty et al., Anal Bioanal Chem 402(2):721-729, 2012; Marty et al., Analytical Chemistry 84:8957-8960, 2012), the entire ND-target can be flown on the MS, and by varying the impact voltage, oligomeric state and lipids can be identified (Keener et al., Anal Chem 93(1):583-597, 2021).

### Example 5: Assessment of the Immunogenicity of M2:ND That Incorporate Immune-Stimulatory Molecules

To enhance the immunogenicity of M2:NDs, the effect of incorporating immune-stimulatory (IS) molecules into the ND assemblies is evaluated. Two IS molecules are evaluated; one is the well-characterized toll-like receptor 4 (TLR4) agonist, monophosphoryl lipid A (MPLA), and the second is a cholesterol anchored TLR9 CpG oligonucleotide agonist (CpG). These IS molecules are included in the overall ND assembly as described below. The co-delivery of these two IS molecules into the same ND assembly as the antigen has been shown to enhance the immunogenicity of the incorporated antigen (Fischer et al., J Am Chem Soc 135(6):2044-2047, 2013). To enable the incorporation of this CpG, a cholesterol-modified ODN D19 is used. MPLA-SM VacciGrade is from InVivoGen. The generation of M2:NDs for immunization trials includes self-assembling M2 into 10 nm ND structures. One or both adjuvants (cholesterol-anchored CpG and MLPA) are co-incorporated into ND assemblies. Adjuvant(s) is added into the carrier lipid (DOPC) in chloroform and then dried to a thin lipid film. The lipid mixture is solubilized in cholate detergent at a ratio of approximately two cholates per lipid, according to the standard protocol (Schuler et al., Method Mol Bio 974:415-433, 2013). Afterwards, the incorporation of M2 in the ND is performed as described above. The average concentration of cholesterol tagged CpG and MPLA is determined using mass spectrometry. It is expected that the amount of incorporated CpG or MPLA will exactly match the ratio present in the initial reconstitution mixture. Extracting of the total lipids and cholesterol anchored nucleotides allows calibrated thin layer chromatography to also determine the ratios of adjuvant to antigen present. Using mass spectrometry to characterize ND and quantitate the absolute stoichiometries of incorporated lipid and protein components is described in Gao et al. (Anal Chem 84(21):8957-8960, 2012).

Bioactivity of adjuvanted M2:ND assemblies in vitro

The bioactivity of the IS molecules incorporated into the M2:ND assemblies is assessed by measuring their ability to stimulate porcine cells to secrete cytokines. To measure the bioactivity of the TLR4 agonist MPLA incorporated into M2:ND (TM2:MPLA:ND), ZMAC cells, a porcine alveolar macrophage line, are exposed for 8 hours to various concentrations of such assemblies and the amount of TNF-α secreted is measured by ELISA. Similarly, to measure the bioactivity of the TLR9 agonist CpG D19 incorporated into M2:ND (M2:CpG:ND), freshly isolated porcine PBMCs are exposed for 18 hours to various concentrations of such assemblies and the amount of IFN-α secreted is measured by ELISA. Plasmacytoid dendritic cells are present among the PBMC population and produce ample amounts of IFN-α when exposed to CpG D19 (Calzada-Nova et al., J Virol 85(6):2703-2713, 2011). The cytokine response of ZMAC cells and PBMC to various concentrations of the M2:MPLA:ND and M2:CpG:ND assemblies, respectively, are compared to the response elicited by equivalent concentrations of free MPLA or CpG. As a negative control, in both cases, M2:ND without incorporated agonists are included at equivalent doses (estimated based on the MSP concentration). A method based on the TNF-α response of ZMAC cells to a whole cell lysate of a saprophytic Mycobacteria was developed and validated to assess the loading of this IS material into various type of nanoparticles. Measurement of the IFN-α response of porcine PBMCs to various IS molecules is performed as previously described (Calzada-Nova et al., J Virol 85(6):2703-2713, 2011). It is expected that either type of IS molecule incorporated into ND assemblies will activate target cells and efficiently stimulate cytokine production.

Immunogenicity of M2:ND assemblies and adjuvanticity of the IS molecules

To test the adjuvanticity of CpG, the M2-specific immune response of 3-week-old swine is measured after the administration of 20 μg of M2 in a 2 ml volume with or without 50 μg of CpG by comparing the immune response to the following vaccine formulations: free M2 (M2), free M2 mixed with CpG (M2+CpG), M2 incorporated into M2:ND, co-administration of M2 incorporated into ND mixed with CpG (M2:ND+CpG), co-delivery of M2 and CpG incorporated into ND (M2:CpG:ND), and as a negative control, empty ND. To test the adjuvanticity of MPLA, the same antigen/adjuvant combination scheme listed above is used except that CpG is substituted by MPLA using a 20 μg dose.

The various vaccine formulations are administered intramuscularly to groups of 3-week old pigs (n=4 per preparation) twice at a 2- to 3-week interval. The magnitude of the humoral and cell-mediated immune responses of swine to intranasal immunization with adjuvanted and unadjuvanted M2:ND assemblies is determined by testing serum, nasal washes and PBMC samples obtained from the vaccinated pigs at 0, 7, 14, 21, 28 and 35 days after the primary immunization. The magnitude of the humoral immune response is assessed by quantitating M2-specific IgG and IgA in serum and nasal wash by ELISA, as show in FIG. 2 and previously described (Larsen et al., Vet Microbiol 74(1-2):117-131, 2000). The capture antigen for the ELISA is the M2 protein bound to the plate via a precoat with anti-His-tag antibody as described in FIG. 2. An ADCC assay is used to measure the effector function of M2-specific IgA present in the mucosal secretions of M2:ND-immunized pigs. The ADCC activity is determined by a flow cytometric assay using two fluorescent dyes to discriminate target from effector cells, and live from dead cells (Zaritskaya et al., Expert Rev Vaccines 9(6):601-616, 2010). The target for ADCC is SD-PJEC, which is labeled with PKH-67, a membrane-labeling dye, to specifically identify the target cells. The cell-mediated immune response is assessed using an IFN-γ ELISPOT to measure the frequency of antigen-specific interferon-γ-secreting cells (IFN-γ-SC) in PBMC as described (Larsen et al., Vet Microbiol 74(1-2):117-131, 2000; Meier et al., Virology 309(1):18-31, 2003). To determine the frequency of M2-specific IFN-γ-SC, the same E. coli-derived M2 used to prepare the M2:NDs is used to stimulate the PBMC. To measure the response of PBMCs to the M2 protein expressed by the virus, the response to MDCK cell-grown IAV is tested. The IAV is A/swine/Indiana/15TOSU0860/2015 (H1N1) strain, which belongs to the HA HI-γ clade and has the M gene segment of the H1N1 pandemic 2009 lineage (Gao et al., J Gen Virol 98(8):2001-2010, 2017). The amino acid sequence of the M2 protein of this isolate is identical to the sequence of the M2 isoform (pdm) that is incorporated into the M2:ND for immunization (SEQ ID NO: 1).

To control for the specificity of T cell response for the M2 viral antigen, the response is measured of the same PBMCs to the MSP protein used to construct the ND as well as an irrelevant antigen, human cytochrome P450 CYP3A4, both of which are produced in E. coli as is the M2 protein. Also tested is the T cell response to the IAV A/swine/Iowa/A01049038/2010 (H1N1) strain, which belongs to the HA clade H1-γ and carries the M gene segment of the TRIG lineage (Gao et al., J Gen Virol 98(8):2001-2010, 2017), and thus does not match the M2 protein in the M2:ND used for immunization, and is therefore not expected to stimulate T cells specific for the M2 of the pandemic lineage. Whatever frequency of IFN-γ-SC is detected in response to either of these three control antigens, is subtracted from the frequency obtained in response to the M2 protein. The doses of the IS substances, MPLA and CpG, were selected based on prior studies indicating that 5-10 μg of MPLA (Sravanthi et al., Journal of Drug Delivery Science and Technology 28 (Supplement C):56-63, 2015; Xia et al., Sci Rep 6:25735, 2016) and 50 μg of CpG (Alcon et al., AAPS J 7(3):e566-71, 2005) when used as adjuvants in the respiratory mucosa are sufficient to attain an adjuvant effect (Fischer et al., J Am Chem Soc 135(6):2044-2047, 2013).

It is expected that the inclusion of either CpG or MPLA into M2:ND will enhance the intensity of the antibody response to M2 as compared to the humoral immune response of swine immunized with unadjuvanted versions of the same assemblies carrying the same dose of antigen.

### Example 6: Immunization of Swine With M2:ND Adjuvanted With Polyphosphazenes and Assessment of Protective Immunity

This example describes a study that can be used to evaluate protection conferred by immunization with M2:ND adjuvanted with the triple adjuvant combination (TriAdj). The adjuvant TriAdj is comprised of three immune-stimulatory substances that significantly enhance the immune response to a co-administered antigen (Wasan et al., Vaccine 7; 37(11):1503-1515, 2019). The three adjuvant molecules included in TriAdj are: the synthetic double stranded RNA polyl:C; a host defense peptide; and polyphosphazene polymers. The polyphosphazene polymer can readily be made to form microparticles that entrap particles and thus is effective for mucosal or intramuscular delivery (Chand et al., Front Bioeng Biotechnol 9:625482, 2021).

### Assessment of the Ability of M2:ND Adjuvanted With TriAdj to Confer Protective Immunity

Three-week old pigs sourced from a swine influenza virus-free swine farm are treated with ceftiofur crystalline antibiotic (EXCEDE™, Pharmacia & Upjohn Company) per manufacturer's recommended dose and randomly allocated into five groups. Each group of animals is housed separately in pens at a suite of a BSL2 containment facility for 1 week prior to immunization. Although the response to vaccines by females tends to be higher than males due to the influence of sex hormones (Giefing-Kroll et al., Aging Cell 14(3):309-321, 2015), since young pigs are being used several months before they reach puberty, it is not expected that gender will influence the results of these experiments. Regardless, half of the pigs in each treatment group are males and the other half are females. All pigs are confirmed seronegative for swine influenza virus by hemagglutination inhibition assays against H1N1 and H3N2 antigens and free from detectable influenza infection by virus isolation from nasal swabs collected seven days before the first vaccination. The M2:ND vaccine consists of 20 μg of M2 displayed in the M2:ND adjuvanted with TriAdj. The vaccine is prepared by mixing the M2:ND with TriAdj in a 2 ml volume. The M2:ND/TriAdj mixture is administered intranasally (IN) to the first group (N=8) and intramuscularly (IM) to a second group (N=8). As a positive control for the vaccination, a third group (N=8) is vaccinated intramuscularly with a WIV vaccine prepared as previously described (Lager and Vincent, Methods Mol Biol 1161:355-361, 2014; Loving et al., J Virol 87(17):9895-9903, 2013) using a dose of 320 HA units of UV-irradiated virus adjuvanted with the o:w adjuvant EMULSIGEN®-D in a 2 ml volume. As a negative control, a fourth group (N-8) is mock vaccinated. The mock vaccine consists of bare NDs (without the M2 protein) mixed with TriAdj and is administered both IN and IM.

The respective vaccines are administered by IM injection or IN using a nasal sprayer twice at a 2- to 3-week interval. Fourteen days after the second vaccination, the animals are challenged intranasally with 106 tissue culture infectious dose 50 (TCID₅₀)/ml of H3N2 swIAV. A fifth group of strict controls pigs (n=4) is not vaccinated or challenged. Pigs are observed daily for clinical signs during the 5 days after challenge. Nasal swabs are obtained at 0, 3 and 5 days after the virus challenge, and serum samples are collected 0, 21, and 35-days post vaccination. Anticoagulated whole blood for isolation of mononuclear cells to assess cell mediated immunity (CMI) are collected 0, 21 and 35-days post vaccination.

All pigs enrolled in the study are humanely euthanized at 5 days after being challenged and their lungs examined for evidence of macroscopic pneumonia, collection of lung lavage fluid (LLF), and collection of lung tissue for evaluation of microscopic lesions. LLF is collected for virus titration as described previously, using 50 ml Minimum Essential Medium per lung lavage (Larsen et al., Vet Microbiol 74(1-2):117-131, 2000; Vincent et al., Vaccine 25(47):7999-8009, 2007). Viral titers are determined using a plaque forming unit assay using MDCK cell monolayers as the substrate for virus replication (Landreth et al., Vet Microbiol 253:108968, 2021). The presence of macroscopic lung lesions for all seven lung lobes is examined and scored as previously described (Halbur et al., Vet Pathol 32(6):648-660, 1995). The efficacy of M2:ND is determined by measuring viral load in the tissues and the development of lung pathology. Evidence of protection is defined as statistically significant reduction in lung lesions and virus titers in LLF and nasal swab samples compared to the non-vaccinated groups.

In view of the many possible implementations to which the principles of the disclosure may be applied, it should be recognized that the illustrated implementations are only examples and should not be taken as limiting the scope of the disclosure. Rather, the scope of the disclosure is defined by the following claims.

1. An immunogenic composition, comprising a full-length influenza A virus matrix 2 (M2) protein, an amphipathic molecule, and at least one phospholipid, wherein the M2 protein, the amphipathic molecule and the at least one phospholipid form a nanodisc.

2. The immunogenic composition of claim 1, wherein:
the amino acid sequence of the full-length M2 protein is at least 95% identical to any one of SEQ ID NOs: 1-8; or
the amino acid sequence of the full-length M2 protein comprises or consists of any one of SEQ ID NOs: 1-8.

3-4. (canceled)

5. The immunogenic composition of claim 1, wherein the full-length M2 protein is fused to a protein tag.

6. (canceled)

7. The immunogenic composition of claim 5, wherein the protein tag is a His tag comprising the amino acid sequence GHHHHHHIEGR (SEQ ID NO: 55), GHHHHHHHDYDIPTTENLYFQG (SEQ ID NO: 56), MGHHHHHHIEGR (SEQ ID NO: 57) or MGHHHHHHHDYDIPTTENLYFQG (SEQ ID NO: 58).

8. The immunogenic composition of claim 1, wherein the amphipathic molecule comprises a protein or polypeptide.

9. The immunogenic composition of claim 8, wherein the protein is a membrane scaffold protein (MSP).

10. The immunogenic composition of claim 9, wherein:
the MSP is a derivative of human or porcine apolipoprotein A1 (Apo-A1);
the MSP is a truncated form of human or porcine Apo-A1;
the amino acid sequence of the MSP is at least 95% identical to any one of SEQ ID NOs: 27-52; and/or
the amino acid sequence of the MSP comprises or consists of any one of SEQ ID NOs: 27-52.

11-14 (canceled)

15. The immunogenic composition of claim 1, wherein the amphipathic molecule comprises an organic polymer, or a natural or synthetic nucleic acid.

16. (canceled)

17. The immunogenic composition of claim 1, wherein the at least one phospholipid comprises a glycerophospholipid, an ether glycerophospholipid, or a sphingophospholipid.

18. The immunogenic composition of claim 17, wherein:
the glycerophospholipid comprises phosphatidyl choline, phosphatidyl ethanolamine, phosphatidyl serine, phosphatidyl inositol, cardiolipin, lysophospholipid, dipalmitoyl-phosphatidylcholine, dimyristoyl phosphatidyl choline, 1-palmitoyl-2-oleoyl-phosphatidyl choline, 1-palmitoyl-2-oleoyl-phosphatidyl serine, 1-palmitoyl-2-oleoyl-phosphatidyl ethanolamine, dihexanoyl phosphatidyl choline, dipalmitoyl phosphatidyl ethanolamine, dipalmitoyl phosphatidyl inositol, dimyristoyl phosphatidyl ethanolamine, dimyristoyl phosphatidyl inositol, dihexanoyl phosphatidyl ethanolamine, dihexanoyl phosphatidyl inositol, 1-palmitoyl-2-oleoyl-phosphatidyl ethanolamine, 1-palmitoyl-2-oleoyl-phosphatidyl inositol, or any combination thereof;
the ether glycerophospholipid comprises 1,2-di-O-phytanyl-sn-glycero-3-phosphocholine, 1,2-di-O-phytanyl-sn-glycero-3-phosphoethanolamine, 1,2-di-O-phytanyl-sn-glycerol, glycerol dialkyl glycerol tetraether, 1,2-di-O-octadecyl-sn-glycero-3-phosphocholine, 1,2-di-O-(9Z-octadecenyl)-sn-glycero-3-phosphocholine, 2-3-diphytanyl-O-sn-glycerol, caldarcheol, isocalarcheol, gentiobiosyl archaeol, archaetidylethanoloamine, gentyobiosyl caldarc haetidylethanoloamine, or any combination thereof;
the sphingophospholipid comprises sphingomyelin; and/or
the phospholipid further comprises cholesterol.

19-21. (canceled)

22. The immunogenic composition of claim 1, further comprising an adjuvant.

23. The immunogenic composition of claim 22, wherein:
the adjuvant is incorporated into the nanodisc;
the adjuvant comprises a toll-like receptor 4 (TLR4) or TLR9 agonist;
the adjuvant comprises polyphosphazene; and/or
the adjuvant comprises polyphosphazene, polyI:C and a host defense peptide.

24. (canceled)

25. The immunogenic composition of claim 23, wherein:
the TLR4 agonists comprises monophosphoryl lipid A (MPLA); or
the TLR9 agonist comprises a CpG oligonucleotide.

26. (canceled)

27. The immunogenic composition of claim 25, wherein the CpG oligonucleotide is modified with cholesterol.

28-29. (canceled)

30. The immunogenic composition of claim 1, further comprising:
a pharmaceutically acceptable carrier; and/or
whole inactivated influenza virus.

31. (canceled)

32. A method of eliciting an immune response against influenza A virus in a subject, comprising administering to the subject an effective amount of the immunogenic composition of claim 1.

33. The method of claim 32, wherein the subject has previously received an influenza virus vaccine, or the subject is further administered an influenza virus vaccine.

34. (canceled)

35. The method of claim 33, wherein the influenza virus vaccine comprises whole inactivated virus.

36. The method of claim 32, wherein the immune response comprises both cell-mediated and humoral immune responses.

37. The method of claim 32, wherein the subject is porcine, or the subject is human.

38. (canceled)


SEQUENCE LISTING (58 sequences)

SEQ ID NO: 1 - protein, 97 aa, synthetic construct
MSLLTEVETPTRSEWECRCSDSSDPLAIAANIIGILHLILWITDRLFFKCIYRRFKYGLKRGPSTEGVPESMREEYQQEQQSAVDVDDGHFVNIELE

SEQ ID NO: 2 - protein, 97 aa, synthetic construct
MSLLTEVETPTRSEWECRCSDSSDPLVIAANIIGILHLILWIADRLFFKCIYRRFKYGLKRGPSTEGVPESMREEYQQEQQSAVDVDDGHFVNIELE

SEQ ID NO: 3 - protein, 97 aa, synthetic construct
MSLLTEVETPTRSEWECRCSDSSDPLVIAANIIGILHLILWITDRLFFKCIYRRFKYGLKRGPSTEGVPESMREEYQQEQQSAVDVDDGHFVNIELE

SEQ ID NO: 4 - protein, 97 aa, synthetic construct
MSLLTEVETPIRNGWECKCNDSSDPLTDAASIIGILHLILWILDRLFFKCIYRRFKYGLQRGPSTEGVPESMREEYRQKQQSAVDVDDGHFVNIVLE

SEQ ID NO: 5 - protein, 97 aa, synthetic construct
MSLLTEVETHIRNGWECKCNDSSDPLIDAASIIGILHLILWILDRLFFKCIYRRFKYGLQRGPSTEGVPESMREEYRQKQQSAVDVDDGHFVNIVLE

SEQ ID NO: 6 - protein, 97 aa, synthetic construct
MSLLTEVETPIRSGWECKCNDSSDPLTDAASIIGILHLILWILDRLFFKCIYRRFKHGLQRGPSTEGVPESMREEYRQEQQSAVDVDDGHFVNIVLE

SEQ ID NO: 7 - protein, 97 aa, synthetic construct
MSLLTEVETPTRNGWECKCNDSSDPLTDAASIIGILHLILWILDRLFFKCIYRRFKYGLQRGPSTEGVPESMREEYRQKQQSAVDVDDGHFVNIVLE

SEQ ID NO: 8 - protein, 97 aa, synthetic construct
MSLLTEVETPIRNGWECRCNDSSDSLIDAASIIGILHLILWILDRLFFKCIYRRFKYGLQRGPSTEGVPESMREEYRQKQQSAVDVDDGHFVNIVLE

SEQ ID NO: 9 - protein, 24 aa, synthetic construct
MHHHHHHSSGVDLGTENLYFQSNA

SEQ ID NO: 10 - protein, 22 aa, synthetic construct
MSLLTEVETPTRSEWECRCSDS

SEQ ID NO: 11 - protein, 24 aa, synthetic construct
SDPLAIAANIIGILHLILWITDRL

SEQ ID NO: 12 - protein, 16 aa, synthetic construct
FFKCIYRRFKYGLKRG

SEQ ID NO: 13 - protein, 35 aa, synthetic construct
PSTEGVPESMREEYQQEQQSAVDVDDGHFVNIELE

SEQ ID NO: 14 - protein, 121 aa, synthetic construct
MHHHHHHSSGVDLGTENLYFQSNAMSLLTEVETPTRSEWECRCSDSSDPLAIAANIIGILHLILWITDRLFFKCIYRRFKYGLKRGPSTEGVPESMREEYQQEQQSAVDVDDGHFVNIELE

SEQ ID NO: 15 - protein, 22 aa, synthetic construct
LKLLDNWDSVTSTFSKLREQLG

SEQ ID NO: 16 - protein, 11 aa, synthetic construct
STFSKLREQLG

SEQ ID NO: 17 - protein, 5 aa, synthetic construct
REQLG

SEQ ID NO: 18 - protein, 22 aa, synthetic construct
PVTQEFWDNLEKETEGLRQEMS

SEQ ID NO: 19 - protein, 11 aa, synthetic construct
KDLEEVKAKVQ

SEQ ID NO: 20 - protein, 22 aa, synthetic construct
PYLDDFQKKWQEEMELYRQKVE

SEQ ID NO: 21 - protein, 22 aa, synthetic construct
PLRAELQEGARQKLHELQEKLS

SEQ ID NO: 22 - protein, 22 aa, synthetic construct
PLGEEMRDRARAHVDALRTHLA

SEQ ID NO: 23 - protein, 22 aa, synthetic construct
PYSDELRQRLAARLEALKENGG

SEQ ID NO: 24 - protein, 22 aa, synthetic construct
ARLAEYHAKATEHLSTLSEKAK

SEQ ID NO: 25 - protein, 11 aa, synthetic construct
PALEDLRQGLL

SEQ ID NO: 26 - protein, 24 aa, synthetic construct
PVLESFKVSFLSALEEYTKKLNTQ

SEQ ID NO: 27 - protein, 201 aa, synthetic construct
MAKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 28 - protein, 212 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 29 - protein, 414 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 30 - protein, 168 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPYSDELRQRLAARLEALKENGGARLAEYHAKATEHLSTLSEKAKPALEDLRQGLLPVLESFKVSFLSALEEYTKKLNTQ

SEQ ID NO: 31 - protein, 168 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSARLAEYHAKATEHLSTLSEKAKPALEDLRQGLLPVLESFKVSFLSALEEYTKKLNTQ

SEQ ID NO: 32 - protein, 201 aa, synthetic construct
MGHHHHHHIEGRLKLLDNWDSVTSTFSKLREQLGPVTQEFWDNLEKETEGLRQEMSPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 33 - protein, 201 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPVLESFKVSFLSALEEYTKKLNTQ

SEQ ID NO: 34 - protein, 392 aa, synthetic construct
MGHHHHHHIEGRLKLLDNWDSVTSTFSKLREQLGPVTQEFWDNLEKETEGLRQEMSPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 35 - protein, 201 aa, synthetic construct
MGHHHHHHHDYDIPTTENLYFQGSVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 36 - protein, 190 aa, synthetic construct
MGHHHHHHHDYDIPTTENLYFQGKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 37 - protein, 179 aa, synthetic construct
MGHHHHHHHDYDIPTTENLYFQGKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 38 - protein, 199 aa, synthetic construct
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLL

SEQ ID NO: 39 - protein, 289 aa, synthetic construct
MGHHHHHHHDYDIPTTENLYFQGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 40 - protein, 278 aa, synthetic construct
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 41 - protein, 423 aa, synthetic construct
MGHHHHHHHDYDIPTTENLYFQGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEYTKKLNTQGTLKLLDNWDSVTSTFSKLREQLGPVTQEFWDNLEKETEGLRQEMKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 42 - protein, 401 aa, synthetic construct
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 43 - protein, 392 aa, synthetic construct
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 44 - protein, 397 aa, synthetic construct
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 45 - protein, 383 aa, synthetic construct
MGHHHHHHHDYDIPTTENLYFQGSVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQNPGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 46 - protein, 379 aa, synthetic construct
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLNPGTKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVTQEFWDNLEKETEGLRQEMS

SEQ ID NO: 47 - protein, 381 aa, synthetic construct
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLSNPGTQKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVTQEFWDNLEKETEGLRQEMS

SEQ ID NO: 48 - protein, 1094 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQSTMGDSHEDTSATMPEAVAEEVSLFSTTDMVLFSLIVGVLTYWFIFRKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDVAKVYTGEMGRLKSYENQKPPFDAKNPFLAAVTANRKLNQGTERHLMHLELDISDSKIRYESGDHVAVYPANDSALVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRAKEPAGENGGRALVPMFVRKSQFRLPFKSTTPVIMVGPGTGIAPFMGFIQERAWLREQGKEVGETLLYYGCRRSDEDYLYREELARFHKDGALTQLNVAFSREQAHKVYVQHLLKRDREHLWKLIHEGGAHIYVCGDARNMAKDVQNTFYDIVAEFGPMEHTQAVDYVKKLMTKGRYSLDVWS

SEQ ID NO: 49 - protein, 214 aa, synthetic construct
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGT

SEQ ID NO: 50 - protein, 212 aa, synthetic construct
MGHHHHHHIEGCLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 51 - protein, 212 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQCLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 52 - protein, 212 aa, synthetic construct
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHACATEHLSTLSEKAKPALEDLRQGLLPVLESFKVSFLSALEEYTKKLNTQ

SEQ ID NO: 53 - protein, 6 aa, synthetic construct
NYNNYK

SEQ ID NO: 54 - protein, 6 aa, synthetic construct
KYNNYK

SEQ ID NO: 55 - protein, 11 aa, synthetic construct
GHHHHHHIEGR

SEQ ID NO: 56 - protein, 22 aa, synthetic construct
GHHHHHHHDYDIPTTENLYFQG

SEQ ID NO: 57 - protein, 12 aa, synthetic construct
MGHHHHHHIEGR

SEQ ID NO: 58 - protein, 23 aa, synthetic construct
MGHHHHHHHDYDIPTTENLYFQG